(19) United States
(12) Patent Application Publication
(10) Pub. No.: US 2013/0317220 A1
(43) Pub. Date: Nov. 28, 2013

(54) NOVEL PROCESSES FOR PREPARING TRIAZOLO[4, 5-d]PYRIMIDINE DERIVATIVES AND INTERMEDIATES THEREOF

(71) Applicants: Vignesh Nair, Kerala (IN); Nikhil Trivedi, Maharashtra (IN); Anil Shahaji Khile, Mumbai (IN); Nitin Sharadchandra Pradhan, Maharashtra (IN)

(72) Inventors: Vignesh Nair, Kerala (IN); Nikhil Trivedi, Maharashtra (IN); Anil Shahaji Khile, Mumbai (IN); Nitin Sharadchandra Pradhan, Maharashtra (IN)

(73) Assignee: Actavis Group Ptc Ehf, Hafnarfjordur (IS)

(21) Appl. No.: 13/996,100

(22) PCT Filed: Dec. 16, 2011
(86) PCT No.: PCT/IB11/03234

§ 371 Date: Aug. 1, 2013

(30) Foreign Application Priority Data
Dec. 20, 2010 (IN) 3868/CHE/2010
Dec. 31, 2010 (IN) 4048/CHE/2010

(51) Int. Cl. C07D 487/04; C07D 317/44; C07D 405/12; C07C 271/24; C07D 239/48

(52) U.S. Cl. CPC C07D 487/04; C07C 271/24; C07D 239/48; C07D 405/12; C07D 317/44
USPC 544/254; 560/27; 544/317; 549/437; 549/438

(57) ABSTRACT

Provided herein is a novel process for the preparation of triazolo[4,5-d]pyrimidine derivatives. Provided particularly herein is a novel, commercially viable and industrially advantageous process for the preparation of highly pure ticagrelor or a pharmaceutically acceptable salt thereof. Provided further herein is a novel process for the preparation of substituted cyclopentanamine derivatives, which are useful intermediates in the preparation of triazolo[4,5-d]pyrimidine compounds. Provided particularly herein is a novel, commercially viable and industrially advantageous process for the preparation of a ticagrelor intermediate, 2-[[(3aR,4S,6R,6aS)-6-amino-2,2-dimethyltetrahydro-3aH-cyclopenta[d][1,3]-dioxol-4-yl]oxy]-1-ethanol.

### CROSS REFERENCE TO RELATED APPLICATIONS

This application claims the benefit of priority to Indian provisional application Nos. 3868/CHE/2010, filed on Dec. 20, 2010; and 4048/CHE/2010, filed on Dec. 31, 2010; which are incorporated herein by reference in their entirety.

### FIELD OF THE DISCLOSURE

The present disclosure relates to novel processes for the preparation of triazolo[4,5-d]pyrimidine derivatives and intermediates thereof. The present disclosure particularly relates to novel, commercially viable and industrially advantageous processes for the preparation of highly pure ticagrelor or a pharmaceutically acceptable salt thereof and its intermediates.

### BACKGROUND

U.S. Pat. Nos. 6,251,910 and 6,525,060 disclose a variety of triazolo[4,5-d]pyrimidine derivatives, processes for their preparation, pharmaceutical compositions comprising the derivatives, and methods of use thereof. These compounds act as P2T (P2YADP or P2TAC) receptor antagonists and they are indicated for use in therapy as inhibitors of platelet activation, aggregation and degranulation, promoters of platelet disaggregation, and anti-thrombotic agents. Among them, Ticagrelor, [1S-(1α,2α,3β(1S*,2R*),5β)]-3-[7-[2-(3,4-difluorophenyl)cyclopropyl]amino]-5-(propylthio)-3H-1,2,3-triazolo[4,5-d]pyrimidin-3-yl)-5-(2-hydroxyethoxy)-cyclopentane-1,2-diol, acts as an adenosine uptake inhibitor, a platelet aggregation inhibitor, a P2Y12 purinoceptor antagonist, and a coagulation inhibitor. It is indicated for the treatment of thrombosis, angina, ischemic heart diseases, and coronary artery diseases. Ticagrelor is the first reversibly binding oral adenosine diphosphate (ADP) receptor antagonist and is chemically distinct from thienopyridine compounds like clopidogrel. It selectively inhibits P2Y12, a key target receptor for ADP. ADP receptor blockade inhibits the action of platelets in the blood, reducing recurrent thrombotic events. The drug has shown a statistically significant primary efficacy against the widely prescribed clopidogrel (Plavix®) in the prevention of cardiovascular (CV) events including myocardial infarction (heart attacks), stroke, and cardiovascular death in patients with acute coronary syndrome (ACS). Ticagrelor is represented by the following structural formula Ia:

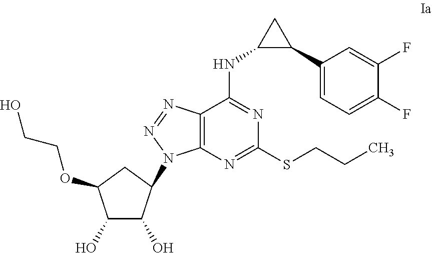 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

According to the '060 patent, ticagrelor is prepared by the condensation of 4,6-dichloro-5-nitro-2-(propylthio)pyrimidine with [3 aR-(3aα,4α,6α,6aα)]-6-amino-tetrahydro-2,2-dimethyl-4H-cyclopenta-1,3-dioxol-4-ol hydrochloride salt in the presence of N,N-diisopropylethylamine in tetrahydrofuran to produce [3aR-(3aα,4α,6α,6aα)]-6-[[6-chloro-5-nitro-2-(propylthio)-pyrimidin-4-yl]amino]-tetrahydro-2,2-dimethyl-4H-cyclopenta-1,3-dioxol-4-ol, followed by reduction in the presence of iron powder in acetic acid to produce [3 aR-(3aα,4α,6α,6aα)]-6-[[5-amino-6-chloro-2-(propylthio)-pyrimidin-4-yl]amino]-tetrahydro-2,2-dimethyl-4H-cyclopenta-1,3-dioxol-4-ol, which is then reacted with isoamyl nitrite in acetonitrile to produce [3aR-(3aα,4α,6α,6aα)]-6-[7-chloro-5-(propylthio)-3H-1,2,3-triazolo[4,5-d]-pyrimidin-3-yl]tetrahydro-2,2-dimethyl-4H-cyclopenta-1,3-dioxol-4-ol. The resulting triazolo[4,5-d]-pyrimidin compound is reacted with ammonia in tetrahydrofuran to produce [3 aR-(3aα,4α,6α,6aα)]-6-[7-amino-5-(propylthio)-3H-1,2,3-triazolo[4,5-d]-pyrimidin-3-yl]tetrahydro-2,2-dimethyl-4H-cyclopenta-1,3-dioxol-4-ol, which is then reacted with a solution of trifluoromethanesulfonyloxy-acetic acid methyl ester in tetrahydrofuran in the presence of butyllithium to produce [3aR-(3aα,4α,6α,6aα)]-6-[[7-amino-5-(propylthio)-3H-1,2,3-triazolo[4,5-d]-pyrimidin-3-yl]tetrahydro-2,2-dimethyl-4H-cyclopenta-1,3-dioxol-4-ol]oxy]acetic acid methyl ester, followed by bromination in the presence of isoamylnitrite in bromoform to produce [3aR-(3aα,4α,6α,6aα)]-6-[[7-bromo-5-(propylthio)-3H-1,2,3-triazolo[4,5-d]-pyrimidin-3-yl]tetrahydro-2,2-dimethyl-4H-cyclopenta-1,3-dioxol-4-ol]oxy]acetic acid methyl ester. The resulting bromo compound is then reacted with (1R-trans)-2-(3,4-difluoro phenyl)cyclopropanamine [R—(R*,R*)]-2,3-dihydroxybutanedioate (1:1) salt in the presence of N,N-diisopropylethylamine in dichloromethane to produce [3aR-[3aα,4α,6α(1R*,2S*),6aα]]-[[6-[7-[[2-(3,4-difluorophenyl)cyclopropyl]amino]-5-(propylthio)-3H-1,2,3-triazolo[4,5-d]-pyrimidin-3-yl]tetrahydro-2,2-dimethyl-4H-cyclopenta-1,3-dioxol-4-ol]oxy]acetic acid methyl ester, followed by reaction with diisobutylaluminium hydride (DIBAL-H) in tetrahydrofuran to produce [3aR-[3aα,4α,6α(1R*,2S*),6aα]]-[[6-[7-[[2-(3,4-difluorophenyl)cyclopropyl]amino]-5-(propylthio)-3H-1,2,3-triazolo[4,5-d]-pyrimidin-3-yl]tetrahydro-2,2-dimethyl-4H-cyclopenta-1,3-dioxol-4-ol]oxy]-ethanol, which is then treated with trifluoroacetic acid in water to produce [1S-(1α,2α,3β(1S*,2R*),5β)]-3-[7-[2-(3,4-difluorophenyl)cyclopropyl]amino]-5-(propylthio)-3H-1,2,3-triazolo[4,5-d]pyrimidin-3-yl)-5-(2-hydroxyethoxy)-cyclopentane-1,2-diol (ticagrelor).

The process for the preparation of ticagrelor disclosed in the '060 patent involves the use of hazardous and explosive materials like DIBAL-H, sodium hydride, isoamyl nitrite and bromoform. The process also involves multiple synthesis steps. The yields of ticagrelor obtained are low to moderate, and the process also involves column chromatographic purifications.

Methods involving column chromatographic purifications are generally undesirable for large-scale operations, thereby making the process commercially unfeasible. Use of explosive reagents like sodium hydride, diazomethane and sodium azide is not advisable, due to the handling difficulties, for scale up operations.

Various processes for the preparation of pharmaceutically active triazolo[4,5-d]pyrimidine cyclopentane compounds, preferably ticagrelor, their enantiomers, and their pharmaceutically acceptable salts are disclosed in U.S. Pat. Nos. 6,251,910; 6,525,060; 6,974,868; 7,067,663; 7,122,695 and 7,250,419; U.S. Patent Application Nos. 2007/0265282, 2008/0132719 and 2008/0214812; European Patent Nos. EP0996621 and EP1135391; and PCT Publication Nos. WO2008/018823 and WO2010/030224.

The processes for the preparation of triazolo[4,5-d]pyrimidine derivatives, preferably ticagrelor and related compounds, described in the above mentioned prior art suffer from disadvantages since the processes involve tedious and cumbersome procedures such as lengthy and multiple synthesis steps, tedious work up procedures, multiple crystallizations or isolation steps, column chromatographic purifications, use of hazardous and/or explosive materials like sodium hydride, isoamyl nitrite, bromoform, diazomethane and sodium azide, and thus resulting in low overall yields of the product. Ticagrelor obtained by the processes described in the aforementioned prior art does not have satisfactory purity. Unacceptable amounts of impurities are formed along with ticagrelor.

One of the useful intermediates in the synthesis of pharmaceutically active triazolo[4,5-d]pyrimidine cyclopentane derivatives is the substituted cyclopentanamine derivative of formula VII:

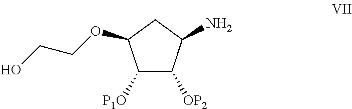

wherein P₁ and P₂ both represents H or a protecting group, or P₁ and P₂ together with the atoms to which they are attached form an alkylidene ring such as a methylidene or isopropylidene ring.

In the preparation of ticagrelor, 2-[[(3aR,4S,6R,6aS)-6-amino-2,2-dimethyltetrahydro-3aH-cyclopenta[d][1,3]-dioxol-4-yl]oxy]-1-ethanol, alternatively named as [3 aR-(3aα,4α,6α,6aα)]-2-[[6-amino-2,2-dimethyltetrahydro-4H-cyclopenta-1,3-dioxol-4-yl]oxy]-ethanol, of formula VIIa:

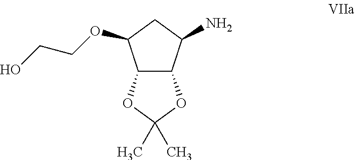

is a key intermediate.

According to the '060 patent, the substituted cyclopentanamine derivatives of formula VII, specifically the [3aR-(3aα,4α,6α,6aα)]-2-[[6-amino-2,2-dimethyltetrahydro-4H-cyclopenta-1,3-dioxol-4-yl]oxy]-ethanol of formula VIIa, is prepared by a process as depicted in scheme 1:

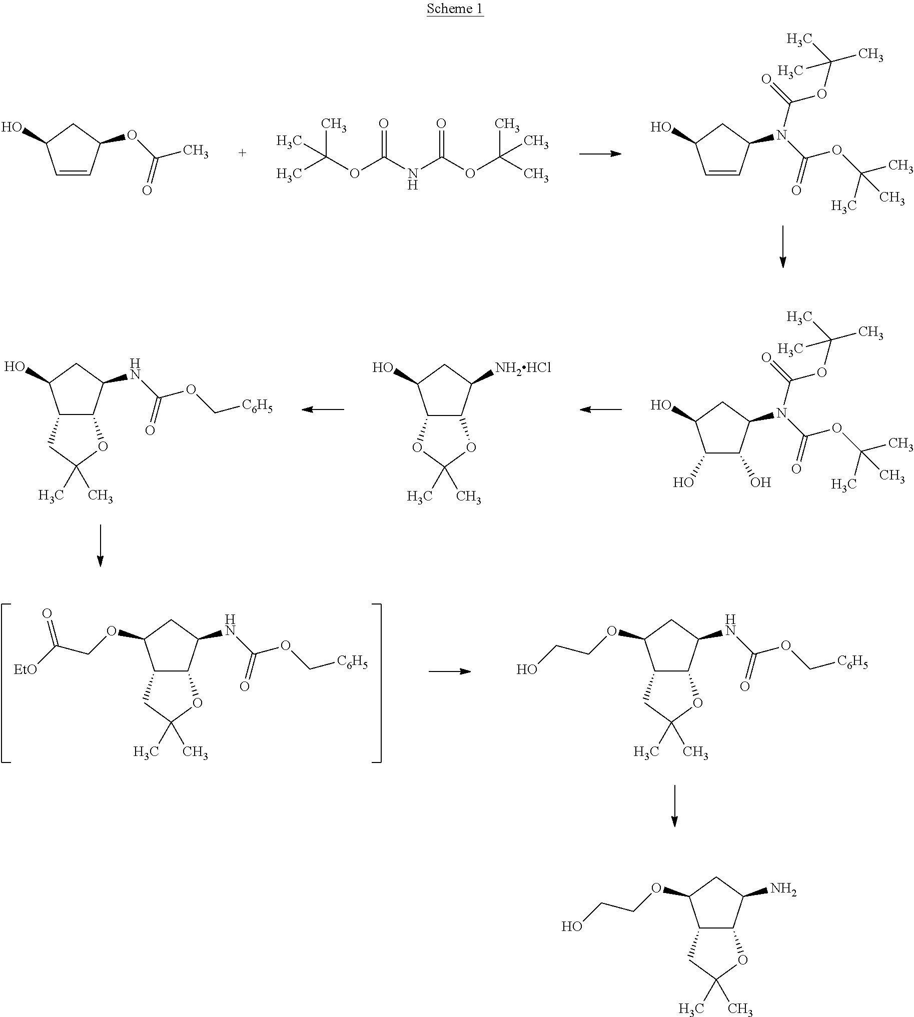

According to the '060 patent, the [3aR-(3aα,4α,6α,6aα)]-2-[[6-amino-2,2-dimethyltetrahydro-4H-cyclopenta-1,3-dioxol-4-yl]oxy]-ethanol is prepared by reacting imidodicarbonic acid bis-(1,1-dimethylethyl)ester with (1S-cis)-4-acetoxy-2-cyclopenten-1-ol in the presence of sodium hydride and tetrakis(triphenylphosphine)palladium in tetrahydrofuran to produce a reaction mass, followed by column chromatographic purification (SiO₂, ethyl acetate:hexane 1:9 as eluant) to produce (1R-cis)-bis(1,1-dimethylethyl)-4-hydroxy-2-cyclopentenylimidodicarbonate, which is then subjected to oxidation in the presence of osmium tetroxide (2.5% solution in tert-butanol) and N-methylmorpholine-N-oxide in a solvent mixture containing tetrahydrofuran and water for 4 days to produce a reaction mass, followed by column chromatographic purification (SiO₂, ethyl acetate:hexane 1:1 as eluant) to produce [1R-(1α,2β,3β,4α)]-2,3,4-trihydroxy-cyclopentenylimidodicarbonic acid, bis(1,1-dimethylethyl)ester. The resulting trihydroxy compound is stirred with hydrochloric acid and methanol for 18 hours to produce a reaction mixture, followed by evaporation to produce a colorless powder, which is then reacted with 2,2-dimethoxypropane and concentrated hydrochloric acid in acetone to produce [3aR-(3aα,4α,6α,6aα)]-6-amino-tetrahydro-2,2-dimethyl-4H-cyclopenta-1,3-dioxol-4-ol hydrochloride salt. The resulting hydroxy compound is then reacted with benzyl chloro formate in the presence of potassium carbonate in 4-methyl-2-pentanone and water to produce a reaction mass, followed by usual work up and subsequent column chromatographic purification (SiO₂, dichloromethane:methanol, 95:5 to 90:10 as eluant) to produce [3aS-(3aα,4α,6α,6aα)]-[tetrahydro-6-hydroxy-2,2-dimethyl-4H-cyclopenta-1,3-dioxol-4-yl]-carbamic acid, phenylmethyl ester. The phenylmethyl ester compound is then reacted with ethyl bromoacetate in the presence of potassium tert-butoxide in tetrahydrofuran to produce a reaction mass containing an ester intermediate, which is, in-situ, subjected to reduction with lithium borohydride in the presence of glacial acetic acid, followed by usual work up and subsequent column chromatographic purification (SiO₂, ethyl acetate:hexane, 25:75 to 50:50 as eluant) to produce [3aS-(3aα,4α,6α,6aα)]-[2,2-dimethyl-6-(2-hydroxyethoxy)-tetrahydro-4H-cyclopenta-1,3-dioxol-4-yl]-carbamic acid, phenylmethyl ester. The resulting hydroxyl compound is then hydrogenated using 5% palladium on charcoal catalyst in ethanol to produce the [3 aR-(3aα,4α,6α,6aα)]-2-[[6-amino-2,2-dimethyltetrahydro-4H-cyclopenta-1,3-dioxol-4-yl]oxy]-ethanol.

The processes for the preparation of 2-[[(3aR,4S,6R,6aS)-6-amino-2,2-dimethyltetrahydro-3aH-cyclopenta[d][1,3]-dioxol-4-yl]oxy]-1-ethanol of formula VII a described in the above mentioned prior art have the following disadvantages and limitations:
a) long reaction times, low yields and low purities of the products;
b) the time required for the oxidation reaction is 4 days, which is industrially not feasible;
c) the processes involve the use of excess amounts of osmium tetroxide, which is an expensive and hazardous reagent, in the oxidation reaction (from about 0.03 equivalents to about 0.12 equivalents with respect to (+)-(1S,4R)-4-phthalimido-2-cyclopenten-1-ol);
d) the processes involve the use of expensive column chromatographic purifications; methods involving column chromatographic purifications are generally undesirable for large-scale operations, thereby making the process commercially unfeasible; and
e) the overall processes generate a large quantity of chemical waste which is difficult to treat.

U.S. Pat. No. 7,393,962 (hereinafter referred to as the '962 patent) discloses a process for the alkylation of substituted cyclopentanamine derivatives by reaction of substituted cyclopentanols with alkyl or arylbromoacetate using metal alkoxide.

The process described in the '962 patent suffers with poor selectivity thus resulting in a poor product quality.

Various processes for syntheses of free amine or hydrochloride salt of substituted cyclopentanoloamine derivatives are apparently disclosed in WO99/05142; Synthetic communications 31 (2001) 18, 2849-2854; Tetrahedron, 1997, 53, 3347; HeIv. Chim. Acta, 1983, 66, 1915; Tetrahedron, 1997, 53, 3347; and Tetrahedron Lett., 2000, 41, 9537.

U.S. Pat. No. 7,067,663; WO2009/064249 and WO2010/030224 disclose L-tartrate, dibenzoyl-L-tartrate and oxalate salts of substituted cyclopentanoloamine derivatives.

Based on the aforementioned drawbacks, the prior art processes have been found to be unsuitable for the preparation of triazolo[4,5-d]pyrimidine derivatives of formula I and substituted cyclopentanamine derivatives of formula VII at lab scale and in commercial scale operations.

A need remains for an improved, commercially viable and industrially advantageous process of preparing triazolo[4,5-d]pyrimidine derivatives, preferably ticagrelor, and their intermediates with high yield and purity, to resolve the problems associated with the processes described in the prior art, and that will be suitable for large-scale preparation. Desirable process properties include use of non-hazardous, environmentally friendly and easy to handle reagents, reduced reaction times, reduced cost, greater simplicity, increased purity, and increased yield of the product, thereby enabling the production of triazolo[4,5-d]pyrimidine compounds, preferably ticagrelor, and their pharmaceutically salts in high purity and in high yield.

### SUMMARY

In one aspect, provided herein is a novel, efficient, industrially advantageous and environmentally friendly process for the preparation of triazolo[4,5-d]pyrimidine derivatives, preferably ticagrelor, or a pharmaceutically acceptable salt thereof, using novel intermediates, in high yield and with high chemical and enantiomeric purity. In another aspect, provided herein is a novel, efficient and industrially advantageous process for the preparation of substituted cyclopentanamine derivatives using novel intermediates, in high yield and with high chemical and enantiomeric purity. In yet another aspect, provided particularly herein is a novel, efficient and industrially advantageous process for the preparation of ticagrelor intermediate, [3 aR-(3aα,4α,6α,6aα)]-2-[[6-amino-2,2-dimethyltetrahydro-4H-cyclopenta-1,3-dioxol-4-yl]oxy]-ethanol, in high yield and with high chemical and enantiomeric purity. Moreover, the processes disclosed herein involve non-hazardous and easy to handle reagents, reduced reaction times and reduced synthesis steps compared to prior art processes. The processes avoid the tedious and cumbersome procedures of the prior art processes and are convenient to operate on a commercial scale.

In another aspect, encompassed herein is the use of novel intermediates or an acid addition salt thereof obtained by the process disclosed herein for preparing ticagrelor or a pharmaceutically acceptable salt thereof.

The processes for the preparation of the triazolo[4,5-d]pyrimidine derivatives and the substituted cyclopentanamine derivatives disclosed herein has the following advantages over the processes described in the prior art:
i) the overall processes involve shorter reaction times and reduced process steps;
ii) the processes avoid the use of hazardous, explosive chemicals like sodium hydride, diazomethane, pyridine and sodium azide;
iii) the processes avoid the use of tedious and cumbersome procedures like column chromatographic purifications and multiple isolations;
iv) the processes avoid the use of expensive materials like chiral sultam auxiliary;
v) the processes involve easy work-up methods and simple isolation processes, and there is a reduction in chemical waste;
vi) the purities of the products are increased without additional purifications; and
vii) the overall yields of the products are increased.

In another aspect, the highly pure [3aR-(3aα,4α,6α,6aα)]-2-[[6-amino-2,2-dimethyltetrahydro-4H-cyclopenta-1,3-dioxol-4-yl]oxy]-ethanol obtained by the process disclosed herein has a total purity, which includes both chemical and enantiomeric purity, of greater than about 95%, specifically greater than about 98%, more specifically greater than about 99%, and most specifically greater than about 99.5% as measured by HPLC.

In another aspect, the present invention also encompasses the use of pure [3 aR-(3aα,4α,6α,6aα)]-2-[[6-amino-2,2-dimethyltetrahydro-4H-cyclopenta-1,3-dioxol-4-yl]oxy]-ethanol obtained by the process disclosed herein for preparing ticagrelor.

### DETAILED DESCRIPTION

According to one aspect, there is provided a process for preparing a triazolo[4,5-d]pyrimidine compound of formula I:

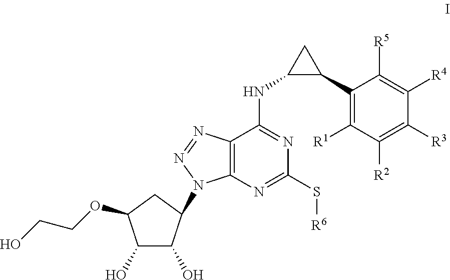

or a pharmaceutically acceptable salt thereof; wherein R¹, R², R³, R⁴ and R⁵ are, each independently, selected from hydrogen and a halogen atom, wherein the halogen atom is F, Cl, Br or I; and R⁶ is C₁₋₆ alkyl; comprising:
a) reacting a substituted phenylcyclopropylamine compound of formula II:

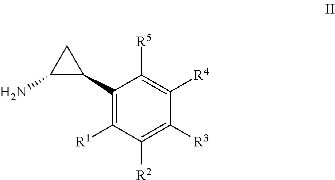

or an acid addition salt thereof, wherein R¹, R², R³, R⁴ and R⁵ are as defined in formula I above, with a compound of formula III:

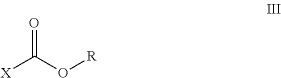

wherein ‘X’ is a leaving group selected from a halogen atom, C₁₋₄ alkoxy and —OC(O)OR⁷, wherein R⁷ is C₁₋₄ alkyl; and R is C₁₋₆ alkyl or benzyl, wherein the phenyl ring of benzyl is optionally substituted by halogen, nitro, S(O)₂(C₁₋₄ alkyl), cyano, C₁₋₄ alkyl, C₁₋₄ alkoxy, C(O)(C₁₋₄ alkyl), N(C₁₋₆alkyl)₂, CF₃ or OCF₃;
in the presence of a first base in a first solvent to produce a carbamic acid ester compound of formula IV:

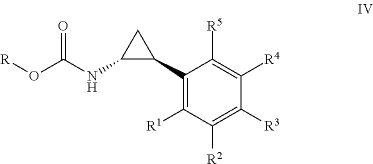

or an acid addition salt thereof, wherein R, R¹, R², R³, R⁴ and R⁵ are as defined above;
b) reacting the carbamic acid ester compound of formula IV with a dichloropyrimidine compound of formula V:

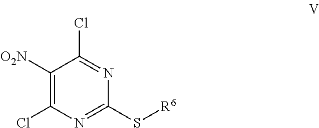

wherein R⁶ is C₁₋₆ alkyl; in the presence of a second base in a second solvent to produce a pyrimidine compound of formula VI:

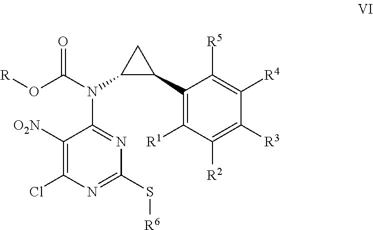

or a pharmaceutically acceptable salt thereof, wherein R, R¹, R², R³, R⁴, R⁵ and R⁶ are as defined above;
c) reacting the compound formula VI with a cyclopentanamine compound formula VII;

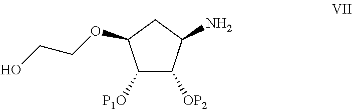

or an acid addition salt thereof, wherein P₁ and P₂ are protecting groups, or P₁ and P₂ together with the atoms to which they are attached form an alkylidene ring such as methylidene or isopropylidene ring;
in the presence of a third base in a third solvent to produce a diaminopyrimidine compound of formula VIII:

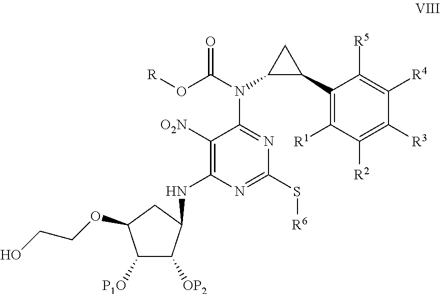

or an acid addition salt thereof, wherein P₁, P₂, R, R¹, R², R³, R⁴, R⁵ and R⁶ are as defined above;
d) reducing the diaminopyrimidine compound formula VIII using a reducing agent in a fourth solvent to produce a triaminopyrimidine compound of formula IX:

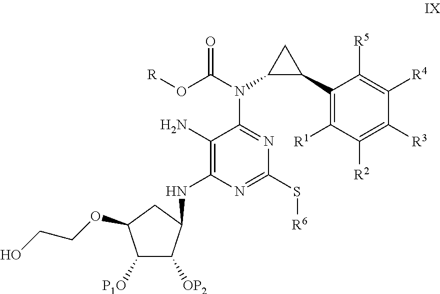

or an acid addition salt thereof, wherein P₁, P₂, R, R¹, R², R³, R⁴, R⁵ and R⁶ are as defined above;
e) reacting the triaminopyrimidine compound of formula IX with a nitrite reagent in a fifth solvent in the presence of an acid to produce a triazol compound of formula X:

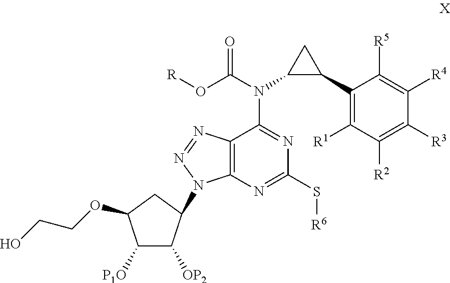

or a pharmaceutically acceptable salt thereof, wherein P₁, P₂, R, R¹, R², R³, R⁴, R⁵ and R⁶ are as defined above; and
f) subjecting the triazol compound of formula X to acid hydrolysis or hydrogenolysis with a suitable acid in a sixth solvent to produce the triazolo[4,5-d]pyrimidine compound of formula I, and optionally converting the compound of formula I obtained into a pharmaceutically acceptable salt thereof.

In one embodiment, the halogen atom as defined in the compounds of formulae I, II, IV, VI, VIII, IX and X is F or Cl; and a more specific halogen atom is F.

In another embodiment, the group ‘R6’ in the compounds of formulae I, V, VI, VIII, IX and X is selected from methyl, ethyl, n-propyl, iso-propyl, n-butyl, iso-butyl and sec.-butyl; and more specifically R⁶ is n-propyl.

In one embodiment, the halogen atom in the compounds of formula III is F, Cl, Br or I; and a more specific halogen atom is Cl.

In another embodiment, the group ‘R’ in the compounds of formula III is selected from methyl, ethyl, n-propyl, iso-propyl, n-butyl, iso-butyl, tert-butyl and sec.-butyl; and more specifically R is tert-butyl.

In another embodiment, the group ‘R7’ in the —OC(O)OR7 as defined for the formula III is selected from methyl, ethyl, n-propyl, iso-propyl, n-butyl, iso-butyl, tert-butyl and sec.-butyl; and more specifically R7 is tert-butyl.

The compounds of formulae I, II, IV, VI, VII, VIII, IX and X can exist in different isomeric forms such as cis/trans isomers, enantiomers, or diastereomers. The process disclosed herein includes all such isomeric forms and mixtures thereof in all proportions unless otherwise specified.

In one embodiment, a most specific triazolo[4,5-d]pyrimidine derivative of formula I prepared by the process described herein is ticagrelor, [1S-(1α,2α,3β(1S*,2R*),5β)]-3-[7-[2-(3,4-difluorophenyl)cyclopropyl]amino]-5-(propylthio)-3H-1,2,3-triazolo[4,5-d]pyrimidin-3-yl)-5-(2-hydroxyethoxy)-cyclopentane-1,2-diol, of formula Ia (formula I, wherein R1, R2 and R5 are H; R3 and R4 are F; and R6 is n-propyl):

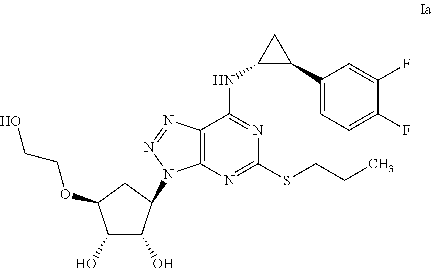 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

or a pharmaceutically acceptable salt thereof.

In another embodiment, a most specific carbamic acid ester compound of formula IV prepared by the process described herein is tert-butyl [(1R,2S)-2-(3,4-difluorophenyl)cyclopropyl]carbamate of formula IVa (formula IV, wherein R1, R² and R⁵ are H; R³ and R⁴ are F; and R is tert-butyl):

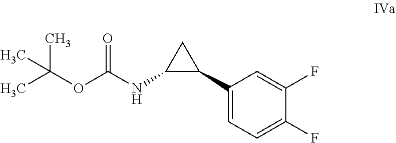 [SMILES: CC(C)(C)OC(=O)N[C@@H]1C[C@H]1c1ccc(F)c(F)c1]

or an acid addition salt thereof.

In another embodiment, a most specific pyrimidine compound of formula VI prepared by the process described herein is 6-chloro-4-[[N-[(1R,2S)-2-(3,4-difluorophenyl)cyclopropan-1-yl]-N-tert-butoxycarbonyl]amino]-5-nitro-2-(propylthio)pyrimidine of formula VIa (formula VI, wherein R¹, R² and R⁵ are H; R³ and R⁴ are F; R is tert-butyl; and R⁶ is n-propyl):

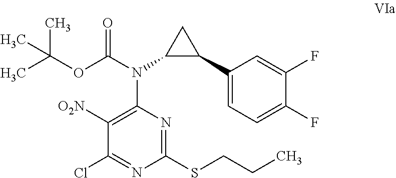 [SMILES: CCCSc1nc(Cl)c([N+](=O)[O-])c(N(C(=O)OC(C)(C)C)[C@@H]2C[C@H]2c2ccc(F)c(F)c2)n1]

or a pharmaceutically acceptable salt thereof.

In another embodiment, a most specific diaminopyrimidine compound of formula VIII prepared by the process described herein is 2-[[(3aR,4S,6R,6aS)-6-[[4-[N-[(1R,2 S)-2-(3,4-difluoro phenyl)cyclopropan-1-yl]-N-tert-butoxycarbonyl]amino]-2-(propylthio)-5-nitro pyrimidin-6-yl]-2,2-dimethyl-tetrahydro-3aH-cyclopenta[d][1,3]dioxol-4-yl]oxy]ethanol of formula VIIIa (formula VIII, wherein R¹, R² and R⁵ are H; R³ and R⁴ are F; R is tert-butyl; R⁶ is n-propyl; and the two groups P₁ and P₂ together with the atoms to which they are attached form an isopropylidene ring):

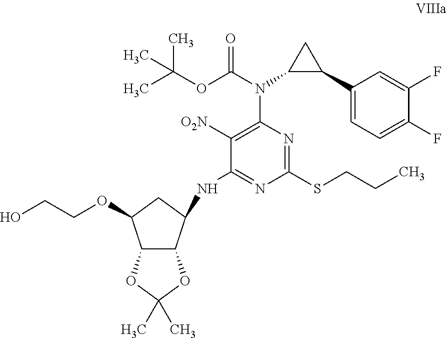 [SMILES: CCCSc1nc(N[C@@H]2C[C@H](OCCO)[C@H]3OC(C)(C)O[C@H]32)c([N+](=O)[O-])c(N(C(=O)OC(C)(C)C)[C@@H]2C[C@H]2c2ccc(F)c(F)c2)n1]

or an acid addition salt thereof.

In another embodiment, a most specific triaminopyrimidine compound of formula IX prepared by the process described herein is 2-[[(3aR,4S,6R,6aS)-6-[[4-[N-[(1R,2S)-2-(3,4-difluoro phenyl)cyclopropan-1-yl]-N-tert-butoxycarbonyl]amino]-2-(propylthio)-5-amino pyrimidin-6-yl]-2,2-dimethyl-tetrahydro-3aH-cyclopenta[d][1,3]dioxol-4-yl]oxy]ethanol of formula IXa (formula IX, wherein R¹, R² and R⁵ are H; R³ and R⁴ are F; R is tert-butyl; R⁶ is n-propyl; and the two groups P₁ and P₂ together with the atoms to which they are attached form an isopropylidene ring):

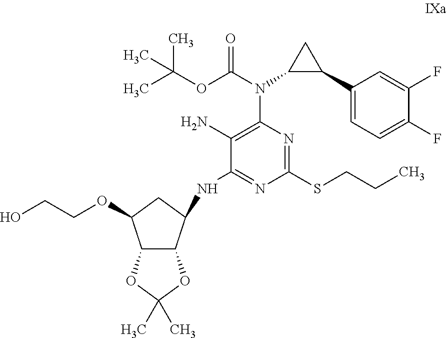 [SMILES: CCCSc1nc(N[C@@H]2C[C@H](OCCO)[C@H]3OC(C)(C)O[C@H]32)c(N)c(N(C(=O)OC(C)(C)C)[C@@H]2C[C@H]2c2ccc(F)c(F)c2)n1]

or an acid addition salt thereof.

In another embodiment, a most specific triazol compound of formula X prepared by the process described herein is 2-[[(3aR,4S,6R,6aS)-6-[7-[[[N-(1R,2S)-2-(3,4-difluorophenyl)-cyclopropan-1-yl]-N-tert-butoxycarbonyl]amino]-5-(propylthio)-3H-[1,2,3]triazolo[4,5-d]pyrimidin-3-yl]-2,2-dimethyl-tetrahydro-3aH-cyclopenta[d][1,3]dioxol-4-yl]oxy]ethanol of formula Xa (formula X, wherein R¹, R² and R⁵ are H; R³ and R⁴ are F; R is tert-butyl; R⁶ is n-propyl; and the two groups P₁ and P₂ together with the atoms to which they are attached form an isopropylidene ring):

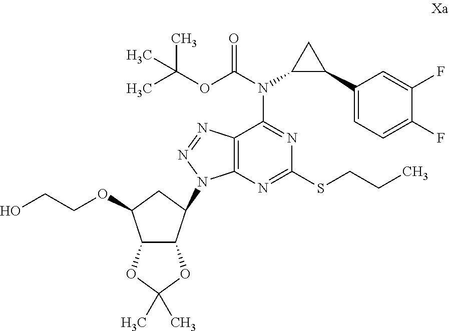 [SMILES: CCCSc1nc(N(C(=O)OC(C)(C)C)[C@@H]2C[C@H]2c2ccc(F)c(F)c2)c2nnn([C@@H]3C[C@H](OCCO)[C@H]4OC(C)(C)O[C@H]43)c2n1]

or a pharmaceutically acceptable salt thereof.

The compounds of formulae IV, VI, VIII, IX and X are novel and constitute another aspect of the disclosure.

Exemplary protecting groups in the compounds of formulae VII, VIII, IX and X are C1-6 alkyl (specifically methyl), benzyl, (C1-6 alkyl)3Si (specifically t-butyldimethylsilyl), and a C(O)C1-6 alkyl group such as acetyl.

In one embodiment, the two groups P1 and P2 together with the atoms to which they are attached form an isopropylidene ring.

Alternatively P1 and P2 can form an alkoxymethylidene ring such as ethoxymethylidene.

Protecting groups can be added and removed using known reaction conditions. The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

In one embodiment, a specific acid addition salt of the substituted phenylcyclopropylamine compound of formula II employed in step-(a) is a mandelate salt, and more specifically (R)-(−)-mandelate salt.

Exemplary first solvents used in step-(a) include, but are not limited to, a ketone, an aliphatic or alicyclic hydrocarbon, a chlorinated aliphatic or aromatic hydrocarbon, an aromatic mono or dinitro hydrocarbon, an aliphatic or cyclic ether, a polar aprotic solvent, and mixtures thereof. The term solvent also includes mixture of solvents.

Specifically, the first solvent is selected from the group consisting of n-pentane, n-hexane, n-heptane, cyclohexane, methylene chloride, dichloroethane, chloroform, carbon tetrachloride, dichlorobenzene, nitrobenzene, dinitrobenzene, tetrahydrofuran, 2-methyl tetrahydrofuran, methyl-tert-butyl ether, diisopropyl ether, methyl cyclopentylether, acetone, methyl ethyl ketone, methyl isobutyl ketone, N,N-dimethylformamide, N,N-dimethylacetamide, N-methylpyrolidone, acetonitrile, and mixtures thereof; and a more specific first solvent is dichloromethane.

Exemplary first bases used in step-(a) include, but are not limited to, sodium hydroxide, sodium bicarbonate, potassium hydroxide, lithium hydroxide, potassium carbonate, sodium carbonate, cesium carbonate, cesium hydroxide, magnesium hydroxide, calcium hydroxide, calcium oxide, tertiary amine bases such as triethyl amine, N,N-diisopropylethylamine, N-methylpiperidine, pyridine, N,N-dimethylaminopyridine, N-methylmorpholine and azabicyclononane.

In one embodiment, the reactions can be homogenous or heterogeneous.

Exemplary compounds of formula III used in step-(a) include, but are not limited to, di-alkyldicarbonates, alkyl chloroformates, substituted aryl dicarbonates and chloroformates. A specific compound of formula III is di-tert-butyldicarbonate.

In one embodiment, the amine protection reaction in step-(a) is carried out at a temperature of about 0° C. to about 100° C., specifically at a temperature of about 20° C. to about 80° C., and more specifically at a temperature of about 40° C. to about 50° C. The reaction time may vary between about 2 hours to about 10 hours, specifically about 3 hours to about 6 hours, and more specifically about 3 hours to about 4 hours.

The reaction mass containing the carbamic acid ester compound of formula IV obtained in step-(a) may be subjected to usual work up such as a washing, an extraction, a pH adjustment, an evaporation or a combination thereof. The reaction mass may be used directly in the next step to produce the compound of formula VI, or the carbamic acid ester compound of formula IV may be isolated and then used in the next step.

In one embodiment, the carbamic acid ester compound of formula IV may be washed with a suitable base to remove acid counter ion. The suitable bases include, but are not limited to, sodium hydroxide, sodium bicarbonate, potassium hydroxide, lithium hydroxide, potassium carbonate, sodium carbonate, cesium carbonate, cesium hydroxide, magnesium hydroxide and calcium hydroxide.

In one embodiment, the carbamic acid ester compound of formula IV is isolated from a suitable solvent by conventional methods such as cooling, seeding, partial removal of the solvent from the solution, by adding an anti-solvent to the solution, evaporation, vacuum drying, spray drying, freeze drying, or a combination thereof.

The solvent used to isolate the carbamic acid ester compound of formula IV is selected from the group consisting of water, aliphatic ether, a hydrocarbon solvent, a chlorinated hydrocarbon, aliphatic alcohols and mixtures thereof. Specifically, the solvent is selected from the group consisting of water, diisopropyl ether, n-heptane, n-pentane, n-hexane, cyclohexane, isopropyl alcohol, n-propyl alcohol and mixtures thereof. A most specific solvent is n-heptane.

Exemplary second bases used in step-(b) include, but are not limited to, metal hydrides such as sodium hydride, lithium hydride, potassium hydride; metal amides such as sodamide, lithium amide, potassium amide; metal alkoxides such as sodium methoxide, potassium tert-butoxide, sodium tert-butoxide, sodium tert-pentoxide, lithium tert-butoxide; alkyl lithium such as n-butyl lithium, n-hexyl lithium; metal diisopropylamide such as lithium diisopropylamide, sodium diisopropyl amide, potassium diisopropyl amide; and metal methylsilazides such as lithium hexamethyldisilazide, sodium hexamethyldisilazide, potassium hexamethyldisilazide.

In one embodiment, the second solvent used in step-(b) is selected from the group consisting of acetone, methyl ethyl ketone, methyl isobutyl ketone, methyl tert-butyl ketone, acetonitrile, tetrahydrofuran, 2-methyl tetrahydrofuran, 1,4-dioxane, diethyl ether, diisopropyl ether, methyl tert-butyl ether, monoglyme, diglyme, n-pentane, n-hexane, n-heptane, cyclohexane, toluene, xylene, N,N-dimethylformamide, N,N-dimethylacetamide, dimethylsulfoxide, N-methylpyrrolidone, and mixtures thereof. A most specific second solvent is tetrahydrofuran.

In one embodiment, the coupling reaction in step-(b) is carried out at a temperature of about −80° C. to about 5° C., specifically at a temperature of about −70° C. to about −20° C., and more specifically at a temperature of about −60° C. to about −50° C. The reaction time may vary between about 30 minutes to about 20 hours, specifically about 1 hour to about 15 hours, and more specifically about 6 hours to about 10 hours. In another embodiment, the reaction mass obtained after completion of the reaction may be quenched by addition of weak acid.

The reaction mass containing the pyrimidine compound of formula VI obtained in step-(b) may be subjected to usual work up methods as described above. The reaction mass may be used directly in the next step, or the compound of formula VI may be isolated, or optionally purified, and then used in the next step.

In one embodiment, the pyrimidine compound of formula VI is isolated and/or purified from a suitable solvent by conventional methods as described above.

The solvent used for isolating or purifying the compound of formula VI is selected from the group consisting of water, an aliphatic ether, a hydrocarbon solvent, a chlorinated hydrocarbon, an aliphatic alcohol, and mixtures thereof. Specifically, the solvent is selected from the group consisting of water, diisopropyl ether, n-heptane, n-pentane, n-hexane, cyclohexane, isopropyl alcohol, n-propyl alcohol, and mixtures thereof.

Exemplary third solvents used in step-(c) include, but are not limited to, a ketone, an aliphatic or alicyclic hydrocarbon, a chlorinated aliphatic or aromatic hydrocarbon, an aromatic mono or dinitro hydrocarbon, an aliphatic or cyclic ether, a polar aprotic solvent, and mixtures thereof. The term solvent also includes mixtures of solvents.

Specifically, the third solvent used in step-(c) is selected from the group consisting of n-pentane, n-hexane, n-heptane, cyclohexane, methylene chloride, dichloro ethane, chloroform, carbon tetrachloride, dichlorobenzene, nitrobenzene, dinitrobenzene, tetrahydrofuran, 2-methyl tetrahydrofuran, methyl-tert-butyl ether, diisopropyl ether, methyl cyclopentylether, acetone, methyl ethyl ketone, methyl isobutyl ketone, N,N-dimethylformamide, N,N-dimethyl acetamide, N-methylpyrolidone, acetonitrile, and mixtures thereof; and a more specific third solvent is tetrahydrofuran.

Exemplary third bases used in step-(c) include, but are not limited to, sodium hydroxide, sodium bicarbonate, potassium hydroxide, lithium hydroxide, potassium carbonate, sodium carbonate, cesium carbonate, cesium hydroxide, magnesium hydroxide, calcium hydroxide, calcium oxide; tertiary amine bases such as triethyl amine, N,N-diisopropylethylamine, N-methylpiperidine, pyridine, N,N-dimethylaminopyridine, N-methylmorpholine and azabicyclononane.

In one embodiment, the reaction in step-(c) is carried out at a temperature of about 0° C. to about 100° C., specifically at a temperature of about 10° C. to about 80° C., and more specifically at a temperature of about 20° C. to about 40° C. The reaction time may vary between about 2 hour to about 10 hours, specifically about 3 hours to about 6 hours, and more specifically about 3 hours to about 4 hours.

The reaction mass containing the diaminopyrimidine compound of formula VIII obtained in step-(c) may be subjected to usual work up methods as described above. The reaction mass may be used directly in the next step to produce the aminopyrimidine compound of formula IX, or the diaminopyrimidine compound of formula VIII may be isolated and then used in the next step.

In one embodiment, the diaminopyrimidine compound of formula VIII is isolated from a suitable solvent by conventional methods such as cooling, seeding, partial removal of the solvent from the solution, by adding an anti-solvent to the solution, evaporation, vacuum drying, spray drying, freeze drying, or a combination thereof.

The solvent used to isolate the diaminopyrimidine compound of formula VIII is selected from the group consisting of water, aliphatic ether, a hydrocarbon solvent, a chlorinated hydrocarbon, aliphatic alcohols and mixtures thereof. Specifically, the solvent is selected from the group consisting of water, diisopropyl ether, n-heptane, n-pentane, n-hexane, cyclohexane, isopropyl alcohol, n-propyl alcohol and mixtures thereof. A most specific solvent is n-heptane.

Exemplary fourth solvents used in step-(d) include, but are not limited to, water, a ketone, an alcohol, a hydrocarbon, a cyclic ether, an aliphatic ether, a chlorinated hydrocarbon, and mixtures thereof.

In one embodiment, the fourth solvent is selected from the group consisting of water, acetone, methyl ethyl ketone, methyl isobutyl ketone, methanol, ethanol, isopropyl alcohol, n-propanol, n-butanol, tetrahydrofuran, 2-methyl tetrahydrofuran, 1,4-dioxane, diethyl ether, diisopropyl ether, methyl tert-butyl ether, dimethoxyethane, diethoxyethane, n-pentane, n-hexane, n-heptane, cyclohexane, toluene, xylene, dichloromethane, dichloroethane, chloroform, and mixtures thereof; and most specifically water, acetone, tetrahydrofuran, and mixtures thereof.

In one embodiment, the reduction in step-(d) is carried out in the presence of an acid or a base.

Exemplary acids employed for reduction include, but are not limited to, mineral acids and organic acids. In one embodiment, the acid is selected from the group consisting of hydrochloric acid, hydrobromic acid, sulfuric acid, acetic acid, propionic acid, butanoic acid, pentanoic acid, hexanoic acid, and mixtures thereof.

Exemplary bases employed for reduction include, but are not limited to, sodium hydroxide, sodium bicarbonate, potassium hydroxide, lithium hydroxide, potassium carbonate, sodium carbonate, cesium carbonate, cesium hydroxide, magnesium hydroxide, calcium hydroxide or calcium oxide, tertiary amine bases such as triethyl amine, N,N-diisopropylethylamine, N-methylpiperidine, pyridine, N,N-dimethylaminopyridine, N-methylmorpholine, and azabicyclononane.

Exemplary reducing agents used in step-(d) include, but are not limited to, noble metal catalysts such as palladium or platinum or its compounds, raney-nickel, ferrous sulfate heptahydrate in aqueous ammonia and the like, and metals such as iron, zinc, cobalt, and mixture thereof. The reduction may be carried out in the presence or absence of hydrogen gas.

In one embodiment, the reduction is carried out by using other reducing agents such as ferric chloride-hydrazine hydrate, sodium dithionite, tin chloride hydrate, tin chloride hydrate-hydrochloric acid, tin-hydrochloric acid, zinc-ammonium formate, zinc-formic acid, zinc-acetic acid, zinc-hydrochloric acid, zinc-hydrazinium monoformate, magnesium-ammonium formate, zinc dust-ammonium chloride, and mixtures thereof. A most specific reducing agent used in step-(d) is sodium dithionite.

In another embodiment, the reduction in step-(d) is carried out by a catalytic hydrogen transfer process. Specifically, the catalytic transfer hydrogenation employs various reagents such as 1,4-cyclohexadiene, cyclohexene, ammonium formate, formic acid, sodium formate, hydrazine, 1,3-cyclohexadiene, trialkylammonium formates, and mixtures thereof. Catalytic transfer hydrogenation reagents are well known, and a selection can be made from these well-known reagents.

In one embodiment, the reduction is carried out at a temperature of about −5° C. to about 80° C. for at least 30 minutes, specifically at a temperature of about 10° C. to about 50° C. for about 1 hour to about 10 hours, and most specifically at about 20° C. to about 40° C. for about 2 hours to about 4 hours.

If necessary, slower addition of the metal catalyst or the acid is employed to minimize the impurity formation. Specifically, the addition time is about 1 hour 30 minutes to about 16 hours, and more specifically about 2 hours to about 5 hours.

The reaction mass containing the triaminopyrimidine compound of formula IX obtained in step-(d) may be subjected to usual work up methods as described above. The reaction mass may be used directly in the next step to produce the triazol compound of formula X, or the triaminopyrimidine compound of formula IX may be isolated and then used in the next step.

In one embodiment, the triaminopyrimidine compound of formula IX is isolated and/or recovered from a suitable solvent by the methods as described above.

The solvent used to isolate the triaminopyrimidine compound of formula IX is selected from the group consisting of water, aliphatic ether, a hydrocarbon solvent, a chlorinated hydrocarbon, aliphatic alcohols and mixtures thereof. Specifically, the solvent is selected from the group consisting of water, diisopropyl ether, n-heptane, n-pentane, n-hexane, cyclohexane, isopropyl alcohol, n-propyl alcohol and mixtures thereof. A most specific solvent is n-heptane.

In one embodiment, the triaminopyrimidine compound of formula IX or an acid addition salt thereof obtained in step-(d) is recovered by techniques such as filtration, filtration under vacuum, decantation, centrifugation, or a combination thereof. In one embodiment, the compound of formula IX is recovered by filtration employing a filtration media of, for example, a silica gel or celite.

Exemplary fifth solvents used in step-(e) include, but are not limited to, water, a hydrocarbon, cyclic ethers, an ether, an ester, a nitrile, an aliphatic amide, a chlorinated hydrocarbon, and mixtures thereof.

In one embodiment, the fifth solvent is selected from the group consisting of water, tetrahydrofuran, 2-methyl tetrahydrofuran, 1,4-dioxane, diethyl ether, diisopropyl ether, methyl tert-butyl ether, dimethoxyethane, diethoxyethane, n-pentane, n-hexane, n-heptane, cyclohexane, toluene, xylene, dichloromethane, dichloroethane, chloroform, ethyl acetate, isopropyl acetate, tert-butyl acetate, acetonitrile, propionitrile, N,N-dimethylformamamide, N,N-dimethylacetamide, and mixtures thereof; and most specifically toluene, water, dichloromethane, 2-methyl tetrahydrofuran, tetrahydrofuran, and mixtures thereof.

Exemplary nitrite reagents used in step-(e) include, but are not limited to, a metal nitrite and an alkyl nitrite, and mixtures thereof.

In one embodiment, the nitrite reagent is selected from the group consisting of sodium nitrite, potassium nitrite, lithium nitrite, butyl nitrite, isoamyl nitrite, and mixtures thereof.

Exemplary acids used in step-(e) include, but are not limited to, mineral acids and organic acids. In one embodiment, the acid is selected from the group consisting of hydrochloric acid, hydrobromic acid, sulfuric acid, acetic acid, propionic acid, butanoic acid, pivalic acid, pentanoic acid, hexanoic acid, methane sulfonic acid, p-toluene sulfonic acid, and mixtures thereof.

In another embodiment, the reaction in step-(e) is carried out at a temperature of about −15° C. to about 50° C. for at least 30 minutes, specifically at a temperature of about −10° C. to about 30° C. for about 1 hour to about 10 hours, and most specifically at about 0° C. to about 10° C. for about 2 hours to about 4 hours.

If necessary, slower addition of the acid is employed to minimize the impurity formation. Specifically, the addition time is about 1 hour 30 minutes to about 16 hours, and more specifically about 2 hours to about 5 hours.

The reaction mass containing the triazol compound of formula X obtained in step-(e) may be subjected to usual work up, followed by isolating and/or recovering from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

The reaction mass may be used directly in the next step to produce the triazolo[4,5-d]pyrimidine compound of formula I, or the triazol compound of formula X may be isolated and then used in the next step.

Exemplary sixth solvents used in step-(f) include, but are not limited to, an alcohol, a hydrocarbon, a cyclic ether, an aliphatic ether, a chlorinated hydrocarbon, and mixtures thereof.

In one embodiment, the sixth solvent is selected from the group consisting of water, methanol, ethanol, isopropyl alcohol, n-propanol, n-butanol, tetrahydrofuran, 2-methyl tetrahydrofuran, 1,4-dioxane, diethyl ether, diisopropyl ether, methyl tert-butyl ether, dimethoxyethane, diethoxyethane, n-pentane, n-hexane, n-heptane, cyclohexane, toluene, xylene, dichloromethane, dichloroethane, chloroform, and mixtures thereof; and most specifically toluene, dichloromethane, 2-methyl tetrahydrofuran, methanol, isopropyl alcohol, tetrahydrofuran, and mixtures thereof.

Exemplary acids used in step-(f) include, but are not limited to, mineral acids and organic acids. In one embodiment, the acid is selected from the group consisting of hydrochloric acid, hydrobromic acid, sulfuric acid, acetic acid, propionic acid, butanoic acid, pivalic acid, pentanoic acid, hexanoic acid, methane sulfonic acid, p-toluene sulfonic acid, camphor sulphonic acid and mixtures thereof; and a most specific acid is hydrochloric acid.

In another embodiment, the reaction in step-(f) is carried out at a temperature of about −15° C. to about 50° C. for at least 30 minutes, specifically at a temperature of about −10° C. to about 40° C. for about 1 hour to about 10 hours, and most specifically at about 0° C. to about 30° C. for about 2 hours to about 4 hours.

If necessary, slower addition of the acid is employed to minimize the impurity formation. Specifically, the addition time is about 1 hour 30 minutes to about 16 hours, and more specifically about 2 hours to about 5 hours.

The reaction mass containing triazolo[4,5-d]pyrimidine compound of formula I obtained in step-(f) may be subjected to usual work up, and followed by isolating and/or recovering from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

The use of inexpensive, non-explosive, non-hazardous, readily available and easy to handle reagents and solvents allows the process disclosed herein to be suitable for preparation of the triazolo[4,5-d]pyrimidine compounds of formula I or an acid addition salt thereof at lab scale and in commercial scale operations.

Acid addition salts of the compounds of formula I can be prepared in high purity by using the substantially pure triazolo[4,5-d]pyrimidine compounds of formula I obtained by the method disclosed herein, by known methods.

The acid addition salts of triazolo[4,5-d]pyrimidine compounds of formula I are derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, tartaric acid, dibenzoyl-L-tartaric acid, di-p-toluoyl-L-tartaric acid, di-p-anisoyl-L-tartaric acid, (R)-(−)-α-methoxyphenyl acetic acid, L-malic acid, (1S)-(+)-10-camphorsulfonic acid, (R) or (S)-α-methoxy-α-(trifluoromethyl)-phenylacetic acid (Mosher's acid), (S) or (R)-(−)-(2-phenylcarbamoyloxy)propionic acid [(S)-(−)-carbamalactic acid], (R) or (S)-para-methylmandelic acid, (R) or (S)-ortho-chloromandelic acid, (R) or (S)-2-hydroxymethylhexanoic acid, (R) or (S)-2-hydroxymethylbutanoic acid and (R) or (S)-2-hydroxymethylpropanoic acid.

Specific acid addition salts of the compounds of formula I are L-tartrate salt, dibenzoyl-L-tartrate salt, di-p-toluoyl-L-tartrate salt, di-p-anisoyl-L-tartrate, (R)-(−)-α-methoxyphenyl acetate, L-malate, (1S)-(+)-10-camphorsulfonate, (R) or (S)-α-methoxy-α-(trifluoromethyl)-phenylacetate, (S) or (R)-(−)-(2-phenylcarbamoyloxy)propionate, (R) or (S)-para-methylmandelate, (R) or (S)-ortho-chloromandelate, (R) or (S)-2-hydroxymethylhexanoate, (R) or (S)-2-hydroxymethylbutanoate and (R) or (S)-2-hydroxymethylpropanoate.

The term “substantially pure triazolo[4,5-d]pyrimidine compound of formula I” refers to the triazolo[4,5-d]pyrimidine compound of formula I, specifically ticagrelor of formula Ia, having a total purity, including both stereochemical and chemical purity, of greater than about 98%, specifically greater than about 99%, more specifically greater than about 99.5%, and still more specifically greater than about 99.9%. The purity is preferably measured by High Performance Liquid Chromatography (HPLC). For example, the purity of the ticagrelor obtained by the process disclosed herein is about 99% to about 99.9%, or about 99.5% to about 99.99%, as measured by HPLC.

The use of the intermediate compounds of formulae IV, VI, VIII, IX, X and their stereochemical isomers, in the preparation of ticagrelor of formula Ia, or a pharmaceutically acceptable acid addition salt thereof, is novel and forms further aspect of the present disclosure.

According to another aspect, there is provided a process for the preparation of a substituted cyclopentanamine derivative of formula VII:

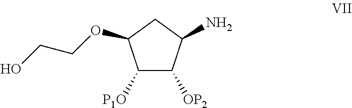

or an acid addition salt thereof; wherein P₁ and P₂ both represents hydrogen or a protecting group, or P₁ and P₂ together with the atoms to which they are attached form an alkylidene ring such as a methylidene or isopropylidene ring; comprising:
a) reacting a cyclopentanol compound of formula XI:

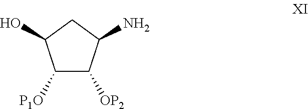

or an acid addition salt thereof, wherein P₁ and P₂ are as defined above, with an alkylating agent of formula XII:

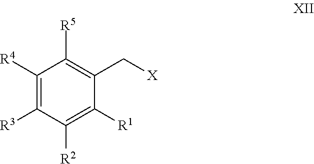

wherein ‘X’ is a leaving group, selected from the group consisting of mesyl, tosyl, Cl, Br and I; and wherein R¹, R², R³, R⁴ and R⁵ are, each independently, selected from hydrogen, F, Cl, Br, I, nitro, C₁-C₃-alkyl, and C₁-C₃-alkoxy substituents; in the presence of a base in a first solvent to produce a benzyl protected compound of formula XIII:

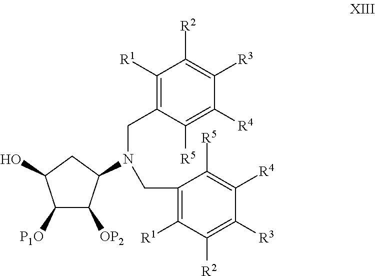

or a pharmaceutically acceptable salt thereof, wherein P₁, P₂, R¹, R², R³, R⁴ and R⁵ are as defined above;
b) reacting the compound of formula XIII with a compound of formula XIV:

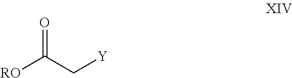

wherein ‘Y’ is a leaving group, selected from the group consisting of mesyl, tosyl, Cl, Br and I; R is C₁₋₆ straight or branched alkyl, or a benzyl group, wherein the phenyl ring of benzyl group is optionally substituted with one or more of the nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃; in the presence of an organic or inorganic base in a second solvent to produce an ester compound of formula XV:

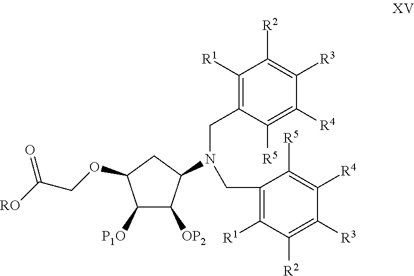

or a pharmaceutically acceptable salt thereof, wherein P₁, P₂, R, R¹, R², R³, R⁴ and R⁵ are as defined above;
c) reducing the ester compound of formula XVI with a reducing agent in the presence of a third solvent to produce a hydroxy compound of formula XVI:

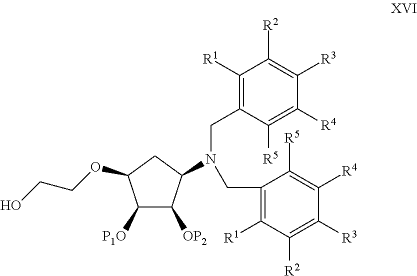

or a pharmaceutically acceptable salt thereof, wherein P₁, P₂, R¹, R², R³, R⁴ and R⁵ are as defined above; and
d) deprotecting the compound of formula XVI in a fourth solvent to produce the substituted cyclopentanamine derivative of formula VII, and optionally converting the compound of formula VII obtained into an acid addition salt thereof.

Exemplary protecting groups in the compounds of formulae VII, XI, XIII, XV and XVI are C₁₋₆ alkyl (preferably methyl), benzyl, (C₁₋₆ alkyl)₃Si (preferably t-butyldimethylsilyl), and a C(O)C₁₋₆ alkyl group such as acetyl.

In one embodiment, the two groups P1 and P2 together with the atoms to which they are attached form an isopropylidene ring.

In another embodiment, the two groups P1 and P2 can form an alkoxymethylidene ring such as ethoxymethylidene.

In one embodiment, the leaving group ‘X’ in the compounds of formula XII is Cl or Br, and more specifically Br.

In another embodiment, the groups R¹, R², R³, R4 and R5 in the compounds of formulae XII, XIII, XV and XVI are hydrogen.

In another embodiment, the leaving group ‘Y’ in the compounds of formula XIV is Cl or Br, and more specifically Br. In another embodiment, the group ‘R’ in the compounds of formulae XIV and XV is tert-butyl.

In one embodiment, a most specific substituted cyclopentanamine derivative of formula VII prepared by the process described herein is [3aR-(3aα,4α,6α,6aα)]-2-[[6-amino-2,2-dimethyl tetrahydro-4H-cyclopenta-1,3-dioxol-4-yl]oxy]-ethanol of formula VIIa (formula VII, wherein P₁ and P₂ together with the atoms to which they are attached form an isopropylidene ring):

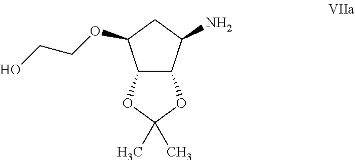

or an acid addition salt thereof.

The compounds of formulae XIII, XV and XVI are novel and constitute another aspect of the invention.

In another embodiment, a most specific benzyl protected compound of formula XIII prepared by the process described herein is (3aR,4S,6R,6aS)-6-(N,N-dibenzylamino)-2,2-dimethyltetrahydro-3aH-cyclopenta[d][1,3]dioxol-4-ol of formula XIIIa (formula XIII, wherein R1, R2, R3, R4 and R⁵ are H; and the two groups P₁ and P₂ together with the atoms to which they are attached form an isopropylidene ring):

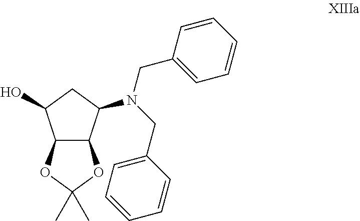 [SMILES: CC1(C)O[C@@H]2[C@H](O1)[C@H](N(Cc1ccccc1)Cc1ccccc1)C[C@@H]2O]

or a pharmaceutically acceptable salt thereof.

In another embodiment, a most specific ester compound of formula XV prepared by the process described herein is tert-butyl [[(3aR,4S,6R,6aS)-6-(N,N-Dibenzylamino)-2,2-dimethyltetrahydro-3aH-cyclopenta[d][1,3]dioxol-4-yl]oxy]acetate of formula XVa (formula XV, wherein R¹, R², R³, R⁴ and R⁵ are H; R is tert-butyl; and the two groups P₁ and P₂ together with the atoms to which they are attached form an isopropylidene ring):

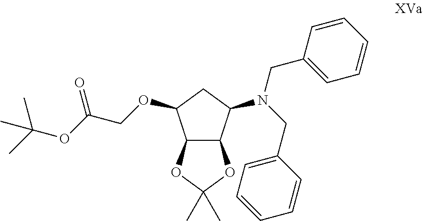 [SMILES: CC(C)(C)OC(=O)CO[C@H]1C[C@@H](N(Cc2ccccc2)Cc2ccccc2)[C@H]2OC(C)(C)O[C@H]21]

or a pharmaceutically acceptable salt thereof.

In another embodiment, a most specific hydroxy compound of formula XVI prepared by the process described herein is 2-[[(3aR,4S,6R,6aS)-6-(N,N-dibenzylamino)-2,2-dimethyltetrahydro-3aH-cyclopenta[d][1,3]dioxol-4-yl]oxy]ethanol of formula XVI a (formula XVI, wherein R¹, R², R³, R⁴ and R⁵ are H; and the two groups P₁ and P₂ together with the atoms to which they are attached form an isopropylidene ring):

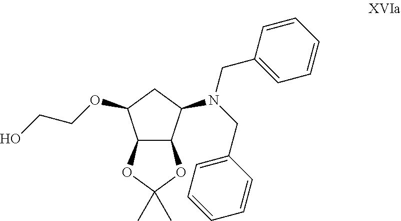 [SMILES: CC1(C)O[C@@H]2[C@H](O1)[C@H](N(Cc1ccccc1)Cc1ccccc1)C[C@@H]2OCCO]

or a pharmaceutically acceptable salt thereof.

Exemplary bases used in step-(a) include, but are not limited to, sodium hydroxide, sodium bicarbonate, potassium hydroxide, lithium hydroxide, potassium carbonate, sodium carbonate, cesium carbonate, cesium hydroxide, magnesium hydroxide, calcium hydroxide, calcium oxide, triethyl amine, N,N-diisopropylethylamine, N-methylpiperidine, pyridine, N,N-dimethylaminopyridine, N-methylmorpholine and azabicyclononane. Specifically, the base is selected from the group consisting of sodium hydroxide, sodium bicarbonate, potassium hydroxide, lithium hydroxide, potassium carbonate and sodium carbonate; and more specifically potassium carbonate.

In one embodiment, the reactions are homogenous or heterogeneous.

Exemplary first solvents used in step-(a) include, but are not limited to, water, a protic solvent, a solvent miscible with water, a dipolar aprotic solvent, and mixtures thereof. The term solvent also includes mixtures of solvents.

Specifically, the first solvent is selected from the group consisting of water, methanol, ethanol, isopropyl alcohol, tetrahydrofuran, acetonitrile, dimethylformamide, dimethylacetamide, tetramethyl urea and its cyclic analog, dimethylsulfoxide, N-methylpyrrolidone, sulfolane, nitromethane, and mixtures thereof; and most specifically a mixture of water and ethanol.

A specific alkylating agent used in step-(a) is benzyl bromide, benzyl chloride, a monosubstituted aralkyl halide or a polysubstituted aralkyl halide. Sulfate or sulfonate esters are also suitable reagents to provide the corresponding benzyl analogs and they can be preformed from the corresponding benzyl alcohol or formed in situ by methods well known to those skilled in the art. Trityl, benzhydryl, substituted trityl, substituted benzhydryl, allyl and substituted allyl groups, independently, are also effective amine protecting groups. Their halide derivatives can also be prepared from the corresponding alcohols by methods well known to those skilled in the art such as treatment with thionyl chloride or bromide, or with phosphorus tri- or penta-halides, or the corresponding phosphoryl trihalide. Examples of groups that can be substituted on the aryl ring include alkyl, alkoxy, acyl, hydroxy, nitro, halo, alkylene, amino, mono- and dialkyl amino, acyl amino, and water solubilizing groups such as phosphonium salts and ammonium salts. The aryl ring can be derived from, for example, benzene, napthelene, indane, anthracene, 9-phenyl-9H-fluorene, durene, phenanthrene and the like. In addition, 1,2-bis(substituted alkylene)-aryl halides or sulfonate esters can be used to form nitrogen containing aryl or non-aromatic heterocyclic derivative or bis-heterocycles. Cycloalkylenealkyl or substituted cyloalkylene radicals containing 6-10 carbon atoms and alkylene radicals constitute additional acceptable class of substituents on nitrogen prepared as outlined above including, for example, cyclohexylenemethylene.

In one embodiment, the alkylation reaction in step-(a) is carried out at a temperature of about 0° C. to about 100° C., specifically at a temperature of about 20° C. to about 80° C., and more specifically at a temperature of about 60° C. to about 70° C. The reaction time may vary between about 2 hours and about 10 hours, specifically about 3 hours and about 6 hours, and more specifically about 3 hours and about 4 hours. The reaction may be carried out under an inert atmosphere such as nitrogen or argon, or normal or dry air, under atmospheric pressure or in a sealed reaction vessel under positive pressure.

Alternatively, the compound of Formula XIII can also be prepared by reductive alkylation by, for example, compounds and intermediates formed from the addition of an aldehyde with the amine and a reducing agent; reduction of a Schiff base, carbinolamine or enamine; or reduction of an acylated amine derivative. Reducing agents include metals (platinum, palladium, palladium hydroxide, palladium on carbon, platinum oxide, rhodium and the like) with hydrogen gas or hydrogen transfer molecules such as cyclohexene or cyclohexadiene; or hydride agents such as lithium aluminum hydride, sodium borohydride, lithium borohydride, sodium cyanoborohydride, diisobutylaluminum hydride or lithium tri-tert-butoxyaluminum hydride.

Additives such as sodium bromide, potassium bromide, sodium iodide and potassium iodide can catalyze or accelerate the rate of amine alkylation, especially when benzyl chloride is used as the nitrogen alkylating agent.

In one embodiment, the reaction in step-(a) is optionally carried out via phase transfer catalysis wherein the amine to be protected and the nitrogen alkylating agent are reacted with a base in a solvent mixture in the presence of a phase transfer reagent, catalyst or promoter. The solvent mixture can consist of, for example, toluene, benzene, ethylene dichloride, cyclohexane, methylene chloride or the like with water, or an aqueous solution of an organic water miscible solvent such as tetrahydrofuran. Exemplary phase transfer catalysts include, but are not limited to, tetrabutylammonium chloride, tetrabutylammonium iodide, tetrabutylammonium bromide, tetrabutylammonium hydroxide, tri-butyloctylammonium chloride, dodecyltrihexylammonium hydroxide, methyltrihexylammonium chloride, and the like.

A specific method of forming substituted amines involves the aqueous addition of about 2 moles of alkylating agent to the amino alcohol. In a more specific method of forming a protected amino alcohol, about 2 moles of benzyl halide in a basic aqueous solution is employed. In a most specific method, the alkylation occurs at 60° C. to 70° C. with potassium carbonate in water, ethanol/water or denatured ethanol/water.

The reaction mass containing the alkylated compound of formula XIII obtained in step-(a) may be subjected to usual work up methods as described above. The reaction mass may be used directly in the next step to produce the compound of formula XV, or the alkylated compound of formula XIII may be isolated and then used in the next step.

In one embodiment, the alkylated compound of formula XIII is isolated from a suitable solvent by the methods as described above.

The solvent used to isolate the alkylated compound of formula XIII is selected from the group consisting of water, tetrahydrofuran, 2-methyl tetrahydrofuran, diisopropyl ether, methyl tert-butyl ether, n-pentane, n-hexane, n-heptane, cyclohexane, toluene, xylene, dichloromethane, dichloroethane, chloroform, and mixtures thereof; and most specifically, toluene, dichloromethane, 2-methyl tetrahydrofuran and mixtures thereof.

In another embodiment, the reaction mass containing the alkylated compound of formula XIII obtained is concentrated and then taken for next step.

Exemplary bases used in step-(b) include, but are not limited to, a metal hydroxide, a metal hydride, a metal amide, a metal alkoxide, an alkyl lithium, a metal diisopropylamide, and a metal methylsilazide.

In one embodiment, the base used in step-(b) is selected from the group consisting of sodium hydroxide, potassium hydroxide, lithium hydroxide, cesium hydroxide, magnesium hydroxide, calcium hydroxide, sodium hydride, lithium hydride, potassium hydride, sodamide, lithium amide, potassium amide, sodium methoxide, potassium tert-butoxide, sodium tert-butoxide, sodium tert-pentoxide, lithium tert-butoxide, n-butyl lithium, n-hexyl lithium, lithium diisopropylamide, sodium diisopropyl amide, potassium diisopropyl amide, lithium hexamethyldisilazide, sodium hexamethyldisilazide and potassium hexamethyldisilazide.

In one embodiment, the second solvent used in step-(b) is selected from the group consisting of acetone, methylethyl ketone, methylisobutyl ketone, methyltert-butyl ketone, acetonitrile, tetrahydrofuran, 2-methyl tetrahydrofuran, 1,4-dioxane, diethyl ether, diisopropyl ether, methyltert-butyl ether, monoglyme, diglyme, n-pentane, n-hexane, n-heptane, cyclohexane, toluene, xylene, N,N-dimethylformamide, N,N-dimethylacetamide, dimethylsulfoxide, N-methylpyrrolidone, and mixtures thereof. A most specific second solvent is N,N-dimethylformamide.

Additives such as sodium bromide, potassium bromide, sodium iodide and potassium iodide can catalyze or accelerate the rate of alkylation reaction, especially when C1 is used as a leaving group in the alkylating agent of formula XIV.

In one embodiment, the reaction in step-(b) is optionally carried out via phase transfer catalysis wherein the alcohol and the alkylating agent are reacted with a base in a solvent mixture in the presence of a phase transfer reagent, catalyst or promoter. The solvent mixture and the phase transfer catalyst are, independently, selected from the group as described above.

In one embodiment, the alkylation reaction in step-(b) is carried out at a temperature of about −50° C. to about 100° C., specifically at a temperature of about −20° C. to about 80° C., and more specifically at a temperature of about 0° C. to about 40° C. The reaction time may vary between about 30 minutes to about 5 hours, specifically about 1 hour to about 4 hours, and more specifically about 2 hours to about 3 hours. In another embodiment, the reaction mass obtained after completion of the reaction may be quenched with water.

The reaction mass containing the alkylated product obtained in step-(b) may be subjected to usual work up methods as described above. The reaction mass may be used directly in the next step, or the compound of formula XV may be isolated, or optionally purified, and then used in the next step.

In one embodiment, the compound of formula XV is isolated and/or purified from a suitable solvent by conventional methods as described above.

Exemplary reducing agents used in step-(c) include, but are not limited to, lithium aluminium hydride, lithium borohydride, sodium borohydride, borane, lithium tri-ter-butoxyaluminum hydride, borane-THF complex, diisobutylaluminum hydride (DIBAL-H), sodium bis(2-methoxyethoxy)aluminum hydride (Vitride®). Specifically, the reducing agent is diisobutylaluminum hydride (DIBAL-H) or sodium bis(2-methoxyethoxy)aluminum hydride (Vitride®) in toluene.

Exemplary third solvents used in step-(c) include, but are not limited to, a hydrocarbon, a cyclic ether, an aliphatic ether, a chlorinated hydrocarbon and the like, and mixtures thereof.

In one embodiment, the third solvent is selected from the group consisting of tetrahydrofuran, 2-methyl tetrahydrofuran, 1,4-dioxane, diethyl ether, diisopropyl ether, methyl tert-butyl ether, n-pentane, n-hexane, n-heptane, cyclohexane, toluene, xylene, dichloromethane, dichloroethane, chloroform, and mixtures thereof; and most specifically, toluene, dichloromethane, 2-methyl tetrahydrofuran, tetrahydrofuran, and mixtures thereof. A most specific third solvent is tetrahydrofuran.

In another embodiment, the reaction in step-(c) is carried out at a temperature of about −20° C. to about 80° C., specifically at a temperature of about −10° C. to about 60° C., and most specifically at about 0° C. to about 35° C. In another embodiment, the reaction is carried out for about 1 hour to about 20 hours, specifically for about 1 hour to about 10 hours, and most specifically for about 1 hour to about 5 hours.

The reaction mass containing the compound of formula XVI obtained in step-(c) may be subjected to usual work up methods as described above. The reaction mass may be used directly in the next step, or the compound of formula XVI may be isolated, or optionally purified, and then used in the next step.

In one embodiment, the compound of formula XVI is isolated and/or purified from a suitable solvent by conventional methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

In one embodiment, the fourth solvent used in step-(d) include, but are not limited to, methanol, ethanol, isopropyl alcohol, n-propanol, n-butanol, tetrahydrofuran, 2-methyl tetrahydrofuran, 1,4-dioxane, diethyl ether, diisopropyl ether, methyl tert-butyl ether, dimethoxyethane, diethoxyethane, toluene, xylene, dichloromethane, dichloroethane, chloroform, and mixtures thereof; and most specifically methanol, ethanol, 2-methyl tetrahydrofuran, tetrahydrofuran, and mixtures thereof.

In one embodiment, the deprotection step comprises the single-step removal of the benzyl protecting groups. The deprotection is carried out either by catalytic hydrogenation in the presence of a hydrogenation catalyst, optionally in the presence of an acid, under high pressure (about 40 to about 100 psi), specifically at a temperature of about 50° C. to about 80° C.; or by catalytic transfer hydrogenation (CTH) in the presence of a catalytic transfer hydrogenation reagent, and optionally in the presence of an acid. Specific hydrogenation catalysts are Pd/C and Pd(OH)₂. A most specific acid is acetic acid.

In another embodiment, the benzyl group can be removed by a catalytic hydrogen transfer process. Specifically, the catalytic transfer hydrogenation reagent is selected from the group consisting of 1,4-cyclohexadiene, cyclohexene, ammonium formate, formic acid, sodium formate, hydrazine, 1,3-cyclohexadiene and trialkylammonium formates, and combinations comprising the foregoing reagents.

In another embodiment, the reaction in step-(d) is carried out at a temperature of about −5° C. to about 80° C. for at least 30 minutes, specifically at a temperature of about 10° C. to about 70° C. for about 1 hour to about 10 hours, and most specifically at about 30° C. to about 60° C. for about 2 hours to about 4 hours.

The reaction mass containing the substituted cyclopentanamine derivative of formula VII or a stereochemically isomeric form or a mixture of stereochemically isomeric forms thereof obtained in step-(d) may be subjected to usual work up, and followed by isolating and/or recovering from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

The use of inexpensive, non-explosive, non-hazardous, readily available and easy to handle reagents and solvents allows the process disclosed herein to be suitable for preparation of the substituted cyclopentanamine derivatives of formula VII or a stereochemically isomeric form or a mixture of stereochemically isomeric forms thereof at lab scale and in commercial scale operations.

Acid addition salts of the compounds of formula VII can be prepared in high purity by using the substantially pure substituted cyclopentanamine derivatives of formula VII or a stereochemically isomeric form or a mixture of stereochemically isomeric forms thereof obtained by the method disclosed herein, by known methods.

The acid addition salts of substituted cyclopentanamine derivatives of formula VII or a stereochemically isomeric form or a mixture of stereochemically isomeric forms thereof are derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, tartaric acid, dibenzoyl-L-tartaric acid, di-p-toluoyl-L-tartaric acid, di-p-anisoyl-L-tartaric acid, (R)-(−)-α-methoxyphenyl acetic acid, L-malic acid, malonic acid, mandelic acid, (1S)-(+)-10-camphorsulfonic acid.

The term “substantially pure substituted cyclopentanamine derivatives” refers to the substituted cyclopentanamine derivatives having a total purity, including both stereochemical and chemical purity, of greater than about 95%, specifically greater than about 98%, more specifically greater than about 99%, and still more specifically greater than about 99.5%. The purity is preferably measured by High Performance Liquid Chromatography (HPLC). For example, the purity of the substituted cyclopentanamine derivatives obtained by the process disclosed herein is about 95% to about 99%, or about 98% to about 99.5%, as measured by HPLC.

Aptly the process for the preparation of the substituted cyclopentanamine derivatives of formula VII disclosed herein is adapted for the preparation of triazolo[4,5-d]pyrimidinecyclopentane compounds, preferably ticagrelor, and their pharmaceutically acceptable acid addition salts, in high enantiomeric and chemical purity.

Ticagrelor and pharmaceutically acceptable acid addition salts thereof can be prepared in high purity by using the substantially pure [3aR-(3aα,4α,6α,6aα)]-2-[[6-amino-2,2-dimethyl tetrahydro-4H-cyclopenta-1,3-dioxol-4-yl]oxy]-ethanol of formula VIIa, by the methods disclosed herein.

The use of the intermediate compounds of formulae XIII, XV and XVI and their stereochemical isomers and acid addition salts thereof, in the preparation of substituted cyclopentanamine derivatives of formula VII or a stereochemically isomeric form or a mixture of stereochemically isomeric forms thereof is novel and forms further aspect of the present disclosure.

In one aspect there is provided a process for the preparation of highly pure compound of formula I and its pharmaceutically acceptable salt.

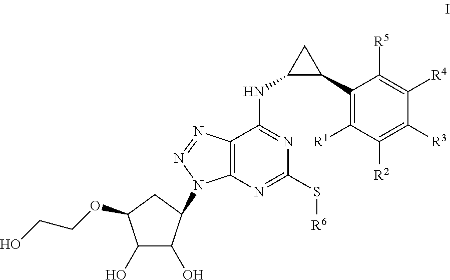

wherein R¹, R², R³, R⁴, R⁵, R⁶ and P¹ and P² are as defined above, comprising o
a) reacting the triazolo compound of formula X

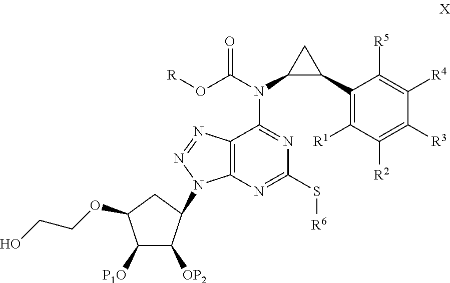

wherein R, R¹, R², R³, R⁴, R⁵, R⁶ and P¹ and P² are as defined above,
with a deprotecting agent in a first solvent to form a compound of formula XVII

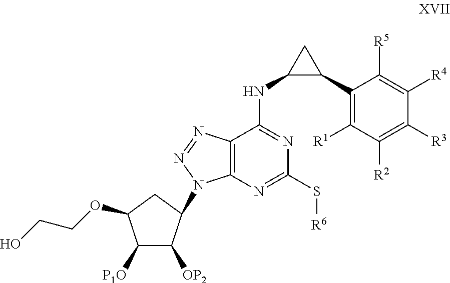

b) reacting a compound of formula XVII with an amino protecting group, in a second solvent and in presence of a base to produce a compound of formula XVIII

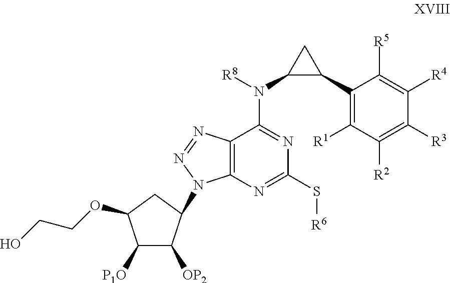

wherein R⁸ is a protecting group
c) reacting the compound of formula XVIII with an acid in a third solvent to produce a compound of formula XIX

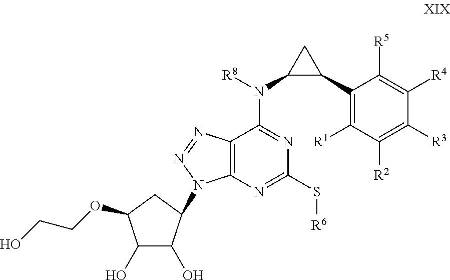

d) treating the compound of formula XIX with a deprotecting agent in a fourth solvent to produce a compound of formula I, and optionally converting the compound of formula I into a pharmaceutically acceptable salt.

In one embodiment, a more specific compound of formula XVII prepared by the process described herein is 2-({(3aR,4S,6R,6aS)-6-[7-{[[N-(1R,2S)-2-(3,4-difluorophenyl)-cyclopropylamino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol of formula XVII (formula XVII, wherein R¹, R² and R⁵ are H; R³ and R⁴ are F; R⁶ is n-propyl; and the two groups P₁ and P₂ together with the atoms to which they are attached form an isopropylidene ring):

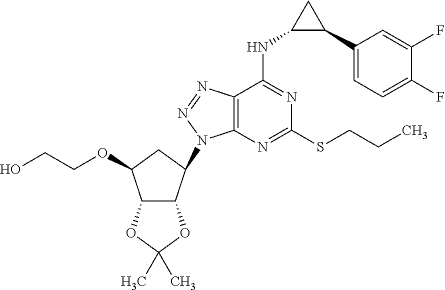 [SMILES: CCCSc1nc(N[C@@H]2C[C@H]2c2ccc(F)c(F)c2)c2nnn([C@@H]3C[C@H](OCCO)[C@H]4OC(C)(C)O[C@H]43)c2n1]

In one embodiment, a more specific compound of formula XVIII prepared by the process described herein is 2-[[(3aR,4S,6R,6 as)-6-[7-[[N-(1R,2S)-2-(3,4-difluorophenyl)-cyclopropylamino]-5-(propylthio)-3H-[1,2,3]triazolo[4,5-d]pyrimidin-3-yl]-2,2-dimethyl-tetrahydro-3aH-cyclopenta[d][1,3]dioxol-4-yl]oxy)ethanol of formula XVIII (formula XVIII, wherein R¹, R² and R⁵ are H; R³ and R⁴ are F; R⁸ is a N-benzyl; R⁶ is n-propyl; and the two groups P₁ and P₂ together with the atoms to which they are attached form an isopropylidene ring):

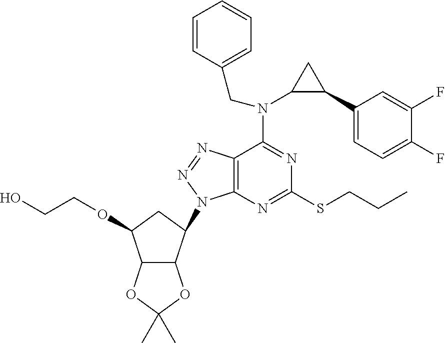 [SMILES: CCCSc1nc(N(Cc2ccccc2)C2C[C@H]2c2ccc(F)c(F)c2)c2nnn([C@@H]3C[C@H](OCCO)C4OC(C)(C)OC43)c2n1]

In one embodiment, a more specific compound of formula XIX prepared by the process described herein is 2-[[(3aR,4S,6R,6 as)-6-[7-[[[N-(1R,2S)-2-(3,4-difluorophenyl)-cyclopropylamino]-5-(propylthio)-3H-[1,2,3]triazolo[4,5-d]pyrimidin-3-yl]-5-(2-hydroxy ethoxy)cyclopentane-1,2-diol of formula XIX (formula XIX, wherein R¹, R² and R⁵ are H; R³ and R⁴ are F; R⁸ is benzyl; R⁶ is n-propyl; and the two groups P₁ and P₂ are independently H);

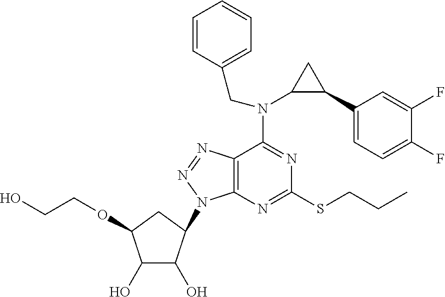 [SMILES: CCCSc1nc(N(Cc2ccccc2)C2C[C@H]2c2ccc(F)c(F)c2)c2nnn([C@@H]3C[C@H](OCCO)C(O)C3O)c2n1]

In one embodiment, the deprotection of compound of formula X in step (a) comprises the single step removal of the protecting group. The deprotection is carried out by the techniques known in the art. More specifically the deprotection step involves adding to the solution of compound of formula X in a solvent, crystals of iodine and heating the reaction mixture to 55 to 60° C. The exemplary first solvent used in step (a) is selected from but not limited to hydrocarbon, ketones, ethers, aliphatic alcohol and mixtures thereof; more specifically the solvent used is acetone.

The reaction mass containing the compound of formula XVII obtained in step (a) may be subjected to usual work up such as washing, an extraction, pH adjustment, an evaporation or a combination thereof.

In another embodiment protecting group introduce in step (b) is selected from any amine protection group. Exemplary protecting groups in the compound of formula XVIII are C1-6alkyl, benzyl, substituted benzyl (C1-6 alkyl)3Si (specifically t-butyldimethylsilyl) and a C(O)C1-6alkyl group.

In another embodiment protecting groups can be added and removed using known reaction conditions. The use of protecting groups is fully described in protective groups in organic chemistry edited by JWF Mcomie, plenum press (1973) and Protective groups in Organic synthesis'2nd Edition, T W Greene & P G M Wiley-interscience.

The second solvent used in step (b) is selected from hydrocarbon, ketones, ethers, aliphatic alcohol and mixtures thereof; more specifically the solvent used is acetone.

The exemplary base used in step (b) is selected from but are not limited to potassium carbonate, sodium carbonate, lithium carbonate and the like; more specifically the base used is potassium carbonate.

In one embodiment the reaction of step (b) takes place at 30 to 100° C. and the reaction time may vary between 10-30 hours; more specifically the reaction takes place at 55 to 60° C. for about 15-20 hours.

In one embodiment the acid hydrolysis of step (c) takes place in a third solvent selected from but not limited to an alcohol, ketone, a hydrocarbon, aliphatic ether, chlorinated hydrocarbon and mixtures thereof.

Exemplary acids used in step (c) include, but are not limited to, mineral acids and organic acids. In one embodiment, the acid is selected from the group consisting of hydrochloric acid, hydrobromic acid, sulfuric acid, acetic acid, propionic acid, butanoic acid, pivalic acid, pentanoic acid, hexanoic acid, methane sulfonic acid and mixtures thereof; and a most specific acid is hydrochloric acid.

In another embodiment, the reaction in step (c) is carried out at a temperature of 0-50° C. for at least 30 minutes

In another embodiment, the pH of the reaction mixture of step (c) is adjusted to between 6-10 with an aqueous base; and most specifically the pH is adjusted to 10 using aqueous potassium carbonate.

In another embodiment the reaction in step (d) involves the deprotection of the amine protecting group. The protecting group used is more specifically a benzyl group. The deprotection step comprises the single-step removal of the protecting groups. The deprotection is carried out either by catalytic hydrogenation in the presence of a hydrogenation catalyst, optionally in the presence of an acid, under high pressure (about 40 to about 100 psi), specifically at a temperature of about 50° C. to about 80° C.; or by catalytic transfer hydrogenation (CTH) in the presence of a catalytic transfer hydrogenation reagent, and optionally in the presence of an acid. Specific hydrogenation catalysts are Pd/C and Pd(OH)₂. A most specific acid is formic acid.

In another embodiment, the benzyl group can be removed by catalytic hydrogen transfer process. Specifically, the catalytic transfer hydrogenation reagent is selected from the group consisting of 1,4-cyclohexadiene, cyclohexene, ammonium formate, formic acid, sodium formate, hydrazine, 1,3-cyclohexadiene and trialkylammonium formates, and combinations comprising the foregoing reagents.

The fourth solvent used in step (d) is selected from but not limited to an alcohol, ketone, a hydrocarbon, aliphatic ether, chlorinated hydrocarbon and mixtures thereof; more specifically the solvent used is ethanol;

In another embodiment, the reaction in step-(d) is carried out at a temperature of about 5° C. to about 80° C. for at least 30 minutes, specifically at a temperature of about 10° C. to about 70° C. for about 1 hour to about 10 hours, and most specifically at about 30° C. to about 60° C. for about 2 hours to about 4 hours.

The reaction mass containing the compound of formula I may be subjected to usual work up, and followed by isolating and/or recovering from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

In another aspect there is provided a process for the preparation of highly pure compound of formula I comprising
a) reacting a triazolo compound of formula X

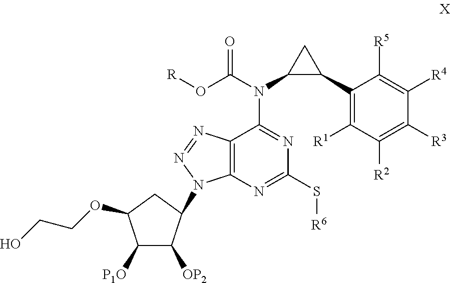

wherein R¹, R², R³, R⁴, R⁵, R⁶ and P₁ and P₂ are as defined above,
with a BOC anhydride in presence of a base to produce a compound of formula XX

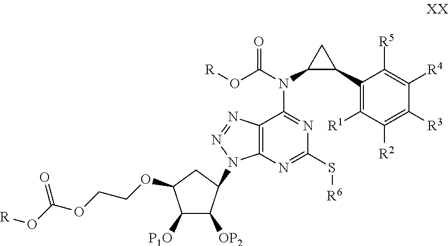

b) subjecting the compound of formula XX to acid hydrolysis or hydrogenolysis with a an acid in a solvent to produce compound of formula I and optionally converting the compound of formula I into a pharmaceutically acceptable salt.

In one embodiment a more specific compound of formula XX prepared by the process described herein is 2-({(3aR,4S,6R,6aS)-6-[7-{[[N-(1R,2S)-2-(3,4-difluorophenyl)-cyclopropan-1-yl]-N-tertbutoxycarbonyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)-O-tert-butoxycarbonylethanol of formula XX a (formula XX, wherein R¹, R² and R⁵ are H; R³ and R⁴ are F; R is tertbutyl; R6 is n-propyl; and the two groups P₁ and P₂ are independently H);

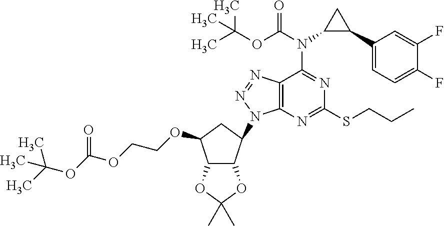

The compounds of formula XVII, XVIII, XIX and XX are novel and constitute another aspects of the disclosure.

In one embodiment the reaction of step (a) involves treating the compound of formula X with a BOC anhydride specifically ditertbutyl dicarbonate.

The exemplary solvent used in step (a) includes, but are not limited to a ketone, an aliphatic or alicyclic hydrocarbon, a chlorinated aliphatic or aromatic hydrocarbon, an aliphatic or cyclic ether, a polar aprotic solvent and mixtures thereof; most specific solvent used is acetone

In another embodiment the reaction in step (a) is carried out at 20-100° C. and the reaction time may vary between 1 hour to 48 hours; more specifically the reaction takes place at a temperature of 20-50° C. for 20-30 hours.

The reaction mass containing the compound of formula XX may be subjected to usual work up, involving the solvent selected from but not limited to water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

The compound of formula XX can be further subjected to recrystallisation using solvents selected from but not limited to water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

Exemplary acids used in step (b) include, but are not limited to, mineral acids and organic acids. In one embodiment, the acid is selected from the group consisting of hydrochloric acid, hydrobromic acid, sulfuric acid, acetic acid, propionic acid, butanoic acid, pivalic acid, pentanoic acid, hexanoic acid, methane sulfonic acid and mixtures thereof; and a most specific acid is hydrochloric acid.

In another embodiment the pH of the reaction mixture of step (c) is adjusted between 6-10 with an aqueous base; and most specifically the pH is adjusted to 10 using aqueous potassium carbonate.

The reaction mass containing the compound of formula I may be subjected to usual work up, and followed by isolating and/or recovering from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

Further encompassed herein is the use of the highly pure ticagrelor or a pharmaceutically acceptable salt thereof obtained by the process disclosed herein for the manufacture of a pharmaceutical composition together with a pharmaceutically acceptable carrier.

In one embodiment, the highly pure ticagrelor or a pharmaceutically acceptable salt thereof obtained by the process disclosed herein has a D90 particle size of less than or equal to about 500 microns, specifically about 1 micron to about 300 microns, and most specifically about 10 microns to about 150 microns.

In another embodiment, the particle sizes of the highly pure ticagrelor or a pharmaceutically acceptable salt thereof are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from thrombosis, angina, Ischemic heart diseases and coronary artery diseases, comprising administering a therapeutically effective amount of the highly pure ticagrelor or a pharmaceutically acceptable salt thereof obtained by the process disclosed herein, or a pharmaceutical composition that comprises a therapeutically effective amount of highly pure ticagrelor or a pharmaceutically acceptable salt thereof, along with pharmaceutically acceptable excipients.

According to another aspect, there is provided a pharmaceutical composition comprising the highly pure ticagrelor or a pharmaceutically acceptable salt thereof prepared according to the processes disclosed herein and one or more pharmaceutically acceptable excipients.

According to another aspect, there is provided a process for preparing a pharmaceutical formulation comprising combining highly pure ticagrelor or a pharmaceutically acceptable salt prepared according to processes disclosed herein, with one or more pharmaceutically acceptable excipients.

Yet in another embodiment, pharmaceutical compositions comprise at least a therapeutically effective amount of highly pure ticagrelor or a pharmaceutically acceptable salt thereof. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure ticagrelor or a pharmaceutically acceptable salt thereof may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure ticagrelor or a pharmaceutically acceptable salt thereof within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

The following examples are given for the purpose of illustrating the present disclosure and should not be considered as limitation on the scope or spirit of the disclosure.

### EXAMPLES

### Example 1

### Preparation of 4,6-Dichloro-5-nitro-2-(propylthio)pyrimidine

### Step-1: Preparation of sodium 2-thiobarbiturate

Dimethyl malonate (500 g) and thiourea (320 g) were added to methanol (1000 ml) under stirring, followed by heating the mixture at reflux temperature (60-65° C.). 30% w/w sodium methoxide solution in methanol (700 g) was slowly added to the hot reaction mass over a period of 30 minutes at reflux temperature (60-65° C.). After completion of the addition, the reaction mass was stirred for 4 hours at reflux temperature (60-65° C.), followed by cooling the mass to 25-30° C. The resulting slurry was stirred for 1 hour at 25-30° C., followed by the isolation of the product by filtration. The resulting wet material was washed with methanol (250 ml). The wet product was dried under reduced pressure at 50-55° C. to produce 521 g of sodium 2-thiobarbiturate as an off white powder (Purity by HPLC: 99.68%).

### Step-2: Preparation of 2-propylthio-pyrimidine-4,6-diol

Sodium-2-thiobarbiturate acid (500 g) was added to a mixture of water (1500 ml) and methanol (1000 ml) under stirring, followed by the addition of n-propyl bromide (407.3 g) at 25-30° C. The resulting mass was stirred for 15 minutes at 25-30° C., followed by the addition of aqueous sodium hydroxide solution (132.44 g in 1500 ml of water) over a period of 6 to 7 hours while maintaining the temperature between 25-30° C. The resulting reaction mixture was stirred for 22 hours at 25-30° C. After completion of the reaction, water (1000 ml) was added to the reaction mass, followed by adjusting the pH of the mass to less than 2 by adding concentrated hydrochloric acid (337 ml). The resulting slurry was stirred for 1 hour and the product was isolated by filtration, followed by washing successively with water (3×1000 ml). The wet product was dried under reduced pressure at 50-55° C. to produce 426.9 g of 2-propylthio-pyrimidine-4,6-diol as a white powder (Purity by HPLC: 94.87%).

### Step-3: Preparation of 5-nitro-2-propylthiopyrimidine-4,6-diol

To a clean and dry reaction assembly containing acetic acid (1000 ml) was added fuming nitric acid (340 ml) over a period of 15 to 20 minutes while maintaining the temperature at 25-30° C. To the mixture was added 2-propylthio-pyrimidine-4,6-diol (400 g) over a period of 60 minutes at 25-30° C., followed by rinsing of the flask with acetic acid (100 ml). The resulting mass was stirred for 1 hour at 25-30° C. After completion of the reaction, water (2400 ml) was added to the mass at 25-30° C. over a period of 20 minutes. The resulting slurry was stirred for 1 hour at 25-30° C. The product was isolated by filtration and then washed successively with water (4×800 ml). The wet product was dried under reduced pressure at 50-55° C. to produce 375 g of 5-nitro-2-propylthiopyrimidine-4,6-diol as a off-white to yellow powder (Purity by HPLC: 99.06%).

### Step-4: Preparation of 4,6-Dichloro-5-nitro-2-(propylthio)pyrimidine

5-Nitro-2-propylthiopyrimidine-4,6-diol (200 g), toluene (1000 ml) and phosphorus oxychloride (425.6 g) were placed in a clean and dry reaction assembly, followed by slow addition of N,N-diisopropylethylamine (230 g) over a period of 30 minutes while maintaining the temperature at below 30° C. The resulting mixture was heated at 110-115° C., followed by maintaining for 3 hours. After completion of the reaction, the reaction mass was cooled to 550° C., followed by distillation of the mixture of toluene and phosphorus oxychloride under reduced pressure. The traces of phosphorus chloride were removed with the addition of toluene (500 ml), followed by evaporation. The resulting mass was diluted with toluene (1000 ml), followed by quenching slowly into water (2000 ml) while maintaining the temperature at below 30° C. and then stirring mixture for 10 minutes. The reaction mass was extracted two times with toluene (1000 ml and 600 ml), followed by washing the organic layer with 3.33% w/v sodium bicarbonate solution (600 ml), 25% w/v sodium chloride solution (600 ml). The toluene layer was stirred with silica gel neutral 60-120 mesh (200 g) and sodium sulfate anhydrous (100 g) for 30 minutes, followed by filtration of the mass through a hyflo bed. The hyflo bed was washed with toluene (2×200 ml) and the washing was combined with the main filtrate. The combined toluene filtrate was evaporated at 50-55° C. under reduced pressure to produce 233.5 g of 4,6-dichloro-5-nitro-2-(propylthio)pyrimidine as an oil (Purity by HPLC: 99.45%).

### Example 2

### Preparation of trans-(1R,2S)-2-(3,4-difluorophenyl)-cyclopropylamine (R)-(−)-mandelate salt

### Step-1: Preparation of 3-Chloro-1-(3′,4′-difluorophenyl)-propan-1-one

1,2-Difluorobenzene (1 kg) was added to a mixture of anhydrous aluminium chloride (1.24 kg) and dichloromethane (1.5 L) under stirring at 20-25° C. The container of 1,2-difluorobenzene was flushed with dichloromethane (0.25 L), followed by adding to the above reaction mass. 3-Chloropropionyl chloride (1.17 kg) was added to the resulting mixture over a period of 60 to 70 minutes while maintaining the temperature at 20-25° C. The container of 3-chloropropionyl chloride was flushed with dichloromethane (0.25 L) and then added to the reaction mass. The resulting mixture was stirred for 30 hours at 20-25° C. After completion of reaction, the reaction mass was quenched into chilled water (10.0 L) while maintaining the temperature at below 25° C. The resulting mixture was extracted with dichloromethane (2×4 L). The combined dichloromethane layer was washed with water (2.5 L), 7% aqueous sodium bicarbonate solution (2.5 L) and water (2×2.5 L). The dichloromethane layer was filtered through a hyflo bed and then the hyflo bed was washed with dichloromethane (2×1.0 L). The filtrate and the washings were combined, followed by concentrating under reduced pressure while maintaining the temperature at below 50° C. The concentrated mass was further degassed to obtain 1.584 kg of 3-chloro-1-(3′,4′-difluorophenyl)-propan-1-one as oil (Yield: 88.34%, Purity by HPLC: 99.10%).

¹H-NMR (CDCl₃, δ): 3.41 (2H, t), 3.91 (2H, t), 7.29 (1H, m), 7.79 (2H, m).

### Step-2: Preparation of 1-(3′,4′-difluorophenyl)-3-nitro-propan-1-one

3-Chloro-1-(3′,4′-difluorophenyl)-propan-1-one (700 g) and N,N-dimethylformamide (1400 ml) were taken into a reaction assembly under nitrogen atmosphere, followed by cooling the mass to 5-10° C. To the resulting suspension was added phloroglucinol (154 g) and sodium iodide (7 g) while maintaining the temperature at about 5-10° C. Sodium nitrite (472.5 g) was added to the resulting mass while maintaining the temperature at about 5-10° C. The resulting reaction mass was stirred for 30 minutes at 5-10° C., followed by raising the mass temperature to 25-30° C. and then maintaining for 3 to 4 hours. After completion of the reaction, the toluene (3500 ml) and water (3500 ml) were added the reaction mass, followed by stirring for 15 minutes. The layers were separated and the aqueous layer was extracted two times with toluene (2×1750 ml). The resulting toluene layers were combined and the combined layer was washed with water (3×2100 ml). The resulting toluene layer was filtered though a hyflo supercel bed and the bed was washed with toluene (2×350 ml). The main filtrate and the washings were combined and the combined filtrate was concentrated to dryness while maintaining the temperature at 50° C. under reduced pressure, followed by co-distillation with isopropyl alcohol (2×350 ml). The resulting mass was dissolved in isopropyl alcohol (2100 ml) at 50-55° C. The resulting clear solution was gradually cooled to 35-45° C., followed by seeding with 1-(3′,4′-difluorophenyl)-3-nitro-propan-1-one (10 g) at 35-40° C. The resulting mass was stirred for 5 hours at 35-40° C., followed by cooling the mass to 20-25° C. The resulting slurry was stirred for 8 to 10 hours at 20-25° C. The resulting slurry was further cooled to −5 to 0° C., followed by stirring for 2 hours at 5 to 0° C. The product was isolated by filtration and then washed two times with chilled isopropyl alcohol (175 and 700 ml). The wet product was dried under reduced pressure at 30-35° C. till the isopropyl alcohol content is less than 1000 ppm to produce 560 g of 1-(3′,4′-difluorophenyl)-3-nitro-propan-1-one (Yield: 76.19%, Purity by HPLC: 99.87%).

### Step-3: Preparation of (1S)-1-(3,4-difluorophenyl)-3-nitropropan-1-ol

Toluene (150 ml), (S)-(−)-2-methyl-CBS-oxazaborolidine solution (1M in toluene, 10 ml) and boron-N,N-diethyl aniline (83.37 g) were taken into a clean and dry reaction assembly at 15-20° C. under nitrogen atmosphere, followed by flushing the assembly with toluene (50 ml). The reaction mass was stirred for 90 minutes at 15-20° C., followed by the addition of a solution of 1-(3′,4′-difluorophenyl)-3-nitro-propan-1-one (100 g) in toluene (250 ml) over a period of 9 to 10 hours at 15-20° C. The addition funnel was flushed with toluene (50 ml) and then added to the reaction mass. The resulting reaction mass was further stirred for 12 hours at 15-20° C. After completion of the reaction, the methanol (50 ml) was added over a period of 30 minutes while maintaining the temperature at below 30° C. The resulting solution was stirred for 30 minutes, followed by the addition of dilute aqueous hydrochloric acid (100 ml of concentrated hydrochloric acid in 400 ml of water). The resulting acidic solution was stirred for 15 minutes, followed by layer separation. The aqueous layer was extracted with toluene (300 ml) and then combined with the main toluene layer. The combined toluene layer was washed twice with dilute aqueous hydrochloric acid (200 ml of concentrated hydrochloric acid in 800 ml of water) and subsequently with water (2×300 ml). The toluene layer was concentrated under reduced pressure to obtain 97.30 g of (1S)-1-(3,4-difluorophenyl)-3-nitropropan-1-ol as an oil (Yield: 96.4%; Purity by HPLC: 97.73%; S-isomer: 96.25%; R-isomer: 3.75%; and [R]²⁵_{D}=+ 37.2° (c 1, CHCl₃)).

### Step-4: Preparation of trans-(1R,2S)-2-(3,4-difluorophenyl)-1-nitrocyclopropane

Triphenyl phosphine (415.16 g) and toluene (825 ml) were taken into a clean and dry reaction assembly and the solution was cooled to 5-10° C., followed by the addition of a solution of diisopropylazodicarboxylate (307.15 g) in toluene (700 ml) over a period of 40 minutes while maintaining the temperature at 5-10° C. After completion of the addition, the addition funnel was rinsed with toluene (125 ml) and then added to the reaction mixture. The resulting solution was stirred for 45 minutes followed by slow addition of a solution of (1S)-1-(3,4-difluorophenyl)-3-nitropropan-1-ol (275 g) in toluene (700 ml) over a period of 1 hour while maintaining the temperature at 5-10° C. After completion of the addition, the addition funnel was rinsed with toluene (125 ml) and then added to the reaction mass. The resulting reaction mass was stirred for 2 hours at 5-10° C. After completion of the reaction, acetic acid (16.5 g) was added to the reaction mass and then stirred for 30 minutes at 5-10° C. The precipitated solid was isolated by filtration and washed with chilled toluene (350 ml). The toluene filtrate and the washings were combined, and the solid cake was discarded. The combined toluene filtrate was washed with dilute aqueous hydrochloric acid (137.5 ml of concentrated hydrochloric acid mixed with 825 ml of water) and 10% aqueous sodium chloride solution (825 ml). The toluene was evaporated at 50-55° C. under reduced pressure to produce crude product as a dark brown oil. The crude product was further purified by distillation under high vacuum to obtain 250 g of trans-(1R,2S)-2-(3,4-difluorophenyl)-1-nitrocyclopropane as a semisolid compound (Yield: 99.2%; Purity by HPLC: 89.99%; [R]²⁵_{D}=191.4° (c 1, CHCl₃)).

### Step-5: Preparation of trans-(1R,2S)-2-(3,4-difluorophenyl)-cyclopropylamine (R)-(−)-mandelate salt

To a pre-cooled methanolic hydrochloric acid solution (6-7% w/w HCl, 4300 ml) was added trans-(1R,2S)-2-(3,4-difluorophenyl)-1-nitrocyclopropane (215 g), and the mixture was cooled to −5 to 0° C. Zinc dust (343.71 g) was added to the resulting mass over a period of 2 to 3 hours while maintaining the temperature at −5 to 0° C. The reaction mass was stirred further for 2 hours at −5 to 0° C. After completion of the reaction, the reaction mass was filtered through a hyflo bed and the bed was washed with methanol (2×215 ml). The main filtrate and the washings were combined, followed by distillation under reduced pressure. The resulting residue was dissolved in dichloromethane (1075 ml) and the solution was cooled to 10 to 15° C. 25% aqueous ammonia solution (1290 ml) was added to the cooled solution while maintaining the temperature at below 30° C. The resulting reaction mass was stirred for 15 minutes, followed by layer separation. The aqueous layer was extracted with dichloromethane (2×537.5 ml), followed by combining with the main dichloromethane layer. The combined dichloromethane layer was extracted thrice with aqueous hydrochloric acid (645 ml of concentrated hydrochloric acid mixed with 1935 ml of water, 3×865 ml). The aqueous acidic layers containing the product were combined, followed by washing with dichloromethane (645 ml). Dichloromethane (1075 ml) and 25% aqueous ammonia solution (1505 ml) were added to the acidic aqueous layer while maintaining the temperature at below 30° C. The resulting reaction mass was extracted twice with dichloromethane (2×645 ml) and then combined with the main dichloromethane layer. The combined dichloromethane layer containing the product was washed with water (645 ml), followed by and evaporation to dryness under reduced pressure. The resulting residue was dissolved in methanol (430 ml), followed slow addition of (R)-(−)-mandelic acid solution (107.5 g in 645 ml methanol) over a period of 40 to 60 minutes while maintaining the temperature at 20 to 25° C. The resulting slurry was stirred further for 12 hours at 20 to 25° C., followed by cooling the slurry to 0 to 5° C. The cooled solution was stirred for 2 hours and the resulting solid was isolated by filtration. The resulting solid was washed with chilled methanol (215 ml). The solid was dried under reduced pressure at 40 to 45° C. to produce 127 g of pure trans-(1R,2S)-2-(3,4-difluorophenyl)-cyclopropylamine (R)-(−)-mandelate salt as a white solid (Purity by HPLC: 99.87%; [R]²⁵_{D}=97.0° (c 1, methanol)).

### Example 3

### Preparation of (3aR,4S,6R,6aS)-6-(N,N-Dibenzylamino)-2,2-dimethyltetrahydro-3aH-cyclopenta[d][1,3]dioxol-4-ol

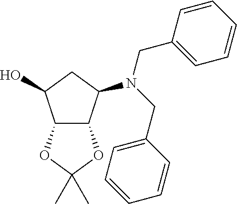 [SMILES: CC1(C)O[C@@H]2[C@H](O1)[C@@H](O)C[C@H]2N(Cc1ccccc1)Cc1ccccc1]

(3aR,4S,6R,6aS)-6-Amino-2,2-dimethyltetrahydro-3aH-cyclopenta[d][1,3]dioxol-4-ol (16 g) was added to a solution of potassium carbonate (44.73 g) in water (64 ml). The resulting suspension was heated at 60-65° C., followed by the addition of benzyl bromide (32.42 g) in ethanol (32 ml) while maintaining the temperature at about 60-65° C. The resulting mixture was stirred for 3 hours at 60-65° C. After completion of the reaction, 25% aqueous ammonia solution (10 ml) was added to the reaction mass, followed by stirring for 15 minutes. The resulting basic solution was extracted twice with toluene (2×75 ml), followed by washing the combined toluene layer with water (75 ml). The toluene layer was concentrated under reduced pressure while maintaining the temperature at below 50° C. The concentrated mass was further purified (silica gel, 30% ethyl acetate in hexane) to produce 27.5 g of (3aR,4S,6R,6aS)-6-(N,N-dibenzylamino)-2,2-dimethyltetrahydro-3aH-cyclopenta[d][1,3]dioxol-4-ol.

¹H-NMR (CDCl₃, δ): 1.31 (3H, s), 1.44 (1H, m), 1.45 (3H, s), 2.14 (2H, dd), 3.23 (1H, m), 3.69 (2H, s), 3.82 (2H, s), 4.09 (1H, m), 4.35 (1H, d), 4.84 (1H, d), 7.23-7.36 (10H, m).

Mass [M+H]: 354.6.

### Example 4

### Preparation of tert-Butyl [[(3aR,4S,6R,6aS)-6-(N,N-Dibenzylamino)-2,2-dimethyl tetrahydro-3aH-cyclopenta[d][1,3]dioxol-4-yl]oxy]acetate

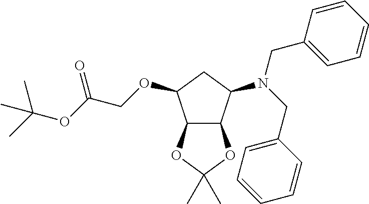 [SMILES: CC(C)(C)OC(=O)CO[C@H]1C[C@@H](N(Cc2ccccc2)Cc2ccccc2)[C@H]2OC(C)(C)O[C@H]21]

A solution of (3aR,4S,6R,6aS)-6-(N,N-dibenzylamino)-2,2-dimethyltetrahydro-3aH-cyclopenta[d][1,3]dioxol-4-ol (25 g) in N,N-dimethylformamide (25 ml) was added to a solution of sodium tert-butoxide (10.2 g) in N,N-dimethylformamide (100 ml) at 0-5° C. over a period of 30 minutes. The resulting solution was stirred for 30 minutes at 0-5° C., followed by the addition of tert-butyl bromoacetate (17.95 g) while maintaining the temperature at about 0-5° C. The resulting mixture was stirred for 2 hours at 0-5° C. After completion of the reaction, water (150 ml) and toluene (200 ml) were added to the reaction mass, followed by stirring for 15 minutes and separating the layers. The aqueous layer was extracted twice with toluene (2×200 ml), followed by washing the combined toluene layer with water (150 ml) and brine solution (150 ml). The toluene layer was concentrated under reduced pressure while maintaining the temperature at below 50° C. The concentrated mass was further purified (silica gel, 24% ethyl acetate in hexane) to produce 17.42 g of tert-Butyl [[(3 aR,4S,6R,6aS)-6-(N,N-Dibenzylamino)-2,2-dimethyltetrahydro-3aH-cyclopenta[d][1,3]dioxol-4-yl]oxy]acetate.

¹H-NMR (CDCl₃, δ): 1.27 (3H, s), 1.42 (3H, s), 1.49 (9H, s), 1.84 (1H, guar), 2.29 (1H, quin), 3.15 (1H, quin), 3.61 (2H, d), 3.72 (2H, d), 3.84 (1H, m), 4.03 (2H, d), 4.43 (1H, m), 4.45 (1H, m), 7.2-7.39 (10H, m). Mass [M+H]: 468.1.

### Example 5

### Preparation of tert-Butyl [[(3aR,4S,6R,6aS)-6-(N,N-Dibenzylamino)-2,2-dimethyl tetrahydro-3aH-cyclopenta[d][1,3]dioxol-4-yl]oxy]acetate

A solution of potassium tert-butoxide in tetrahydrofuran (14.8 ml, 1M) was added to a solution of (3aR,4S,6R,6aS)-6-(N,N-dibenzylamino)-2,2-dimethyltetrahydro-3aH-cyclopenta[d][1,3]dioxol-4-ol (3.5 g) in tetrahydrofuran (17.5 ml) at −5 to 10° C. The resulting solution was stirred for 30 minutes at −5 to 10° C., followed by the addition of a solution of tert-butyl bromoacetate (2.9 g) dissolved in tetrahydrofuran (3.5 ml) while maintaining the temperature between at about −5 to −10° C. The resulting mixture was stirred for 2 hours at −5 to −10° C. After completion of the reaction, a 20% aqueous solution of ammonium chloride (25 ml) was added, followed by stirring for 15 minutes. The layers were separated and the aqueous layer was extracted twice with toluene (2×250 ml), followed by washing the combined organic layer with water (25 ml). The organic layer was dried over sodium sulfate and concentrated under reduced pressure while maintaining the temperature at below 50° C. The concentrated mass was further purified (silica gel, 24% ethyl acetate in hexane) to obtain 3.8 g of tert-Butyl [[(3aR,4S,6R,6aS)-6-(N,N-Dibenzylamino)-2,2-dimethyl tetrahydro-3aH-cyclopenta[d][1,3]dioxol-4-yl]oxy]acetate.

### Example 6

### Preparation of 2-[[(3aR,4S,6R,6aS)-6-(N,N-Dibenzylamino)-2,2-dimethyltetrahydro-3aH-cyclopenta[d][1,3]dioxol-4-yl]oxy]ethanol

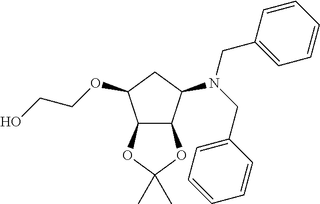 [SMILES: CC1(C)O[C@@H]2[C@H](O1)[C@H](N(Cc1ccccc1)Cc1ccccc1)C[C@@H]2OCCO]

A solution of DIBAL-H (25%, 1M in toluene, 73 ml) was added slowly to a solution of tert-butyl [[(3 aR,4S,6R,6aS)-6-(N,N-dibenzylamino)-2,2-dimethyltetrahydro-3aH-cyclopenta[d][1,3]dioxol-4-yl]oxy]acetate (17 g) in toluene (85 ml) at −20 to −25° C. over a period of 30 minutes. The resulting mixture was stirred for 2 hours at −20 to −25° C. After completion of the reaction, methanol (6 ml) was added to the reaction mass, followed by stirring for 15 minutes. Water (120 ml) and ethyl acetate (90 ml) were added to the resulting solution, followed by the addition of acetic acid (40 ml) and sodium chloride (10 g). The resulting mixture was stirred for 10 minutes, followed by layer separation. The aqueous layer was extracted with ethyl acetate (50 ml), followed by washing the combined organic layer with brine solution (100 ml). The organic layer was dried over sodium sulfate and concentrated under reduced pressure while maintaining the temperature at below 50° C. The concentrated mass was further purified (silica gel, 24% ethyl acetate in hexane) to produce 13 g of 2-[[(3 aR,4S,6R,6aS)-6-(N,N-Dibenzylamino)-2,2-dimethyltetrahydro-3aH-cyclopenta[d][1,3]dioxol-4-yl]oxy]ethanol.

Mass [M+H]: 398.1.

### Example 7

### Preparation of 2-[[(3 aR,4S,6R,6aS)-6-(N,N-Dibenzylamino)-2,2-dimethyltetrahydro-3aH-cyclopenta[d][1,3]dioxol-4-yl]oxy]ethanol

Lithium borohydride (0.28 g) was added to the solution of tert-butyl [[(3aR,4S,6R,6aS)-6-(N,N-dibenzylamino)-2,2-dimethyltetrahydro-3aH-cyclopenta[d][1,3]dioxol-4-yl]oxy]acetate (2 g) in tetrahydrofuran (20 ml) at 20 to 25° C. The resulting mixture was stirred for 2 hours at 20 to 25° C., followed by further stirring for 2 hours at 55 to 60° C. After completion of reaction, methanol (2 ml) was added to the reaction mass, followed by stirring for 15 minutes. 20% aqueous sodium chloride solution (25 ml) was added to the resulting solution, followed by stirring for 5 minutes and then separating of the layers. The aqueous layer was extracted with ethyl acetate (2×25 ml), followed by washing the combined organic layer with a brine solution (25 ml). The organic layer was dried over sodium sulfate and concentrated under reduced pressure to produce 1.5 g of 2-[[(3aR,4S,6R,6aS)-6-(N,N-Dibenzylamino)-2,2-dimethyltetrahydro-3aH-cyclopenta[d][1,3]dioxol-4-yl]oxy]ethanol.

### Example 8

### Preparation of 2-[[(3aR,4S,6R,6aS)-6-Amino-2,2-dimethyltetrahydro-3aH-cyclopenta[d][1,3]-dioxol-4-yl]oxy]-1-ethanol

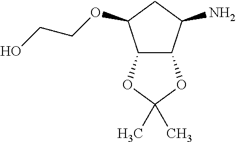

A mixture of 2-[[(3aR,4S,6R,6aS)-6-(N,N-Dibenzylamino)-2,2-dimethyltetrahydro-3aH-cyclopenta[d][1,3]dioxol-4-yl]oxy]ethanol (1.25 g), palladium hydroxide (20% on carbon, 0.3 g) and methanol (150 ml) was taken into an autoclave, followed by nitrogen flushing. The mixture was hydrogenated under hydrogen pressure of 45 psi for 10 hours at 20-25° C. After completion of the reaction, the reaction mass was filtered through a celite bed and the celite bed was washed with methanol (15 ml). The filtrate was concentrated under reduced pressure to obtain 0.7 g of 2-[[(3aR,4S,6R,6aS)-6-amino-2,2-dimethyltetrahydro-3aH-cyclopenta[d][1,3]-dioxol-4-yl]oxy]-1-ethanol.

Mass [M+H]: 218.0

### Example 9

### Preparation of 2-[[(3aR,4S,6R,6aS)-6-Amino-2,2-dimethyltetrahydro-3aH-cyclopenta[d][1,3]-dioxol-4-yl]oxy]-1-ethanol

A mixture of 2-[[(3aR,4S,6R,6aS)-6-(N,N-Dibenzylamino)-2,2-dimethyltetrahydro-3aH-cyclopenta[d][1,3]dioxol-4-yl]oxy]ethanol (17 g), palladium on carbon (10% on carbon, 5 g) and methanol (250 ml) was taken into an autoclave, followed by nitrogen flushing. The mixture was hydrogenated under hydrogen pressure of 35-40 psi for 2 hours at 20-25° C.

After completion of the reaction, the reaction mass was filtered through a celite bed and the bed was washed with methanol (100 ml). The filtrate was concentrated under reduced pressure to produce 8.4 g of 2-[[(3aR,4S,6R,6aS)-6-amino-2,2-dimethyltetrahydro-3aH-cyclopenta[d][1,3]-dioxol-4-yl]oxy]-1-ethanol.

### Example 10

### Preparation of Ticagrelor

### Step-1: Preparation of tert-butyl [(1R,2S)-2-(3,4-difluorophenyl)cyclopropyl]carbamate

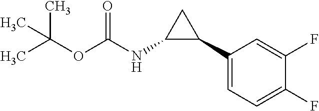 [SMILES: CC(C)(C)OC(=O)N[C@@H]1C[C@H]1c1ccc(F)c(F)c1]

A mixture of trans-(1R,2S)-2-(3,4-difluorophenyl)-cyclopropylamine (R)-(−)-mandelate salt (100 g, prepared according to the example 2), dichloromethane (700 ml) and N,N-diisopropyl ethylamine (45.1 g) was stirred for 30 minutes at 20 to 25° C. To the resulting suspension was added a solution of di-tert-butyl dicarbonate (75.39 g) in dichloromethane (300 ml) over a period of 30 to 40 minutes while maintaining the temperature at 20-25° C. The reaction mass was stirred further for 3 hours at 20-25° C. After completion of the reaction, water (300 ml) was added to the reaction mass. The resulting reaction mass was stirred for 10 minutes, followed by layer separation. The dichloromethane layer containing the product was washed with aqueous potassium carbonate solution (15 g in 300 ml water) and water (300 ml). The dichloromethane layer containing the product was evaporated to dryness under reduced pressure. The resulting residue was dissolved in n-heptane (700 ml) at 80 to 85° C. The resulting clear solution was cooled to 20 to 25° C. and then stirred for 2 hours at 20 to 25° C., followed by isolation of the product by filtration. The resulting solid was washed with n-heptane (200 ml). The solid was dried under reduced pressure at 35 to 40° C. to produce 79 g of tert-butyl [(1R,2S)-2-(3,4-difluorophenyl)cyclopropyl]carbamate (Yield: 94.51%; Purity by HPLC: 99.15%). [¹H-NMR (CDCl₃, δ): 1.1 (2H, m), 1.46 (9H, s), 2.0 (1H, m), 2.65 (1H, m), 4.87 (1H, bs), 6.91-7.28 (3H, m)].

### Step-2: Preparation of 6-Chloro-4-[[N-[(1R,2S)-2-(3,4-difluorophenyl)cyclopropan-1-yl]-N-tert-butoxycarbonyl]amino]-5-nitro-2-(propylthio)pyrimidine

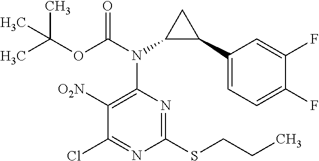 [SMILES: CCCSc1nc(Cl)c([N+](=O)[O-])c(N(C(=O)OC(C)(C)C)[C@@H]2C[C@H]2c2ccc(F)c(F)c2)n1]

To a solution of tert-butyl [(1R,2S)-2-(3,4-difluorophenyl)cyclopropyl]carbamate (5 g) in tetrahydrofuran (50 ml) was slowly added a solution of lithium hexamethyldisilazide (1M in tetrahydrofuran, 25 ml) while maintaining the temperature at about 15 to 25° C. over a period of 30 minutes, followed by stirring the reaction mixture at the same temperature for 30 minutes. The resulting solution was added to a solution of 4,6-dichloro-5-nitro-2-(propylthio)pyrimidine (5.48 g, prepared according to the example 1) in tetrahydrofuran (50 ml) while maintaining the temperature at about 15 to 25° C. over a period of 30 minutes, followed by stirring the mass for 1 hour at the same temperature. After completion of the reaction, saturated ammonium chloride solution (100 ml) was added to the reaction mass. The resulting reaction mass was stirred for 5 minutes, followed by the layer separation. The organic layer containing the product was washed with saturated sodium chloride solution (50 ml) and then dried over sodium sulfate. The organic layer containing the product was evaporated to dryness under reduced pressure. The concentrated mass was further purified (silica gel, 10% ethyl acetate in hexane) to produce 5 g of 6-chloro-4-[[N-[(1R,2S)-2-(3,4-difluorophenyl)cyclopropan-1-yl]-N-tert-butoxycarbonyl]amino]-5-nitro-2-(propylthio)pyrimidine. [¹H-NMR (CDCl₃, δ): 0.84 (2H, m), 1.01 (3H, t), 1.44 (9H, s), 1.71 (2H, m), 2.22 (1H, m), 3.0 (3H, m), 6.92-7.14 (3H, m)].

### Step-3: Preparation of 2-[[(3aR,4S,6R,6aS)-6-[[4-[N-[(1R,2S)-2-(3,4-difluorophenyl)cyclopropan-1-yl]-N-tert-butoxycarbonyl]amino]-2-(propylthio)-5-nitropyrimidin-6-yl]-2,2-dimethyl-tetrahydro-3aH-cyclopenta[d][1,3]dioxol-4-yl]oxy]ethanol

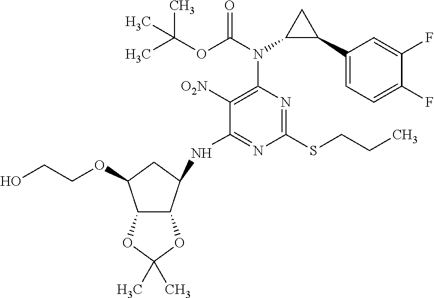 [SMILES: CCCSc1nc(N[C@@H]2C[C@H](OCCO)[C@H]3OC(C)(C)O[C@H]32)c([N+](=O)[O-])c(N(C(=O)OC(C)(C)C)[C@@H]2C[C@H]2c2ccc(F)c(F)c2)n1]

To a solution of 2-[[(3aR,4S,6R,6aS)-6-amino-2,2-dimethyltetrahydro-3aH-cyclopenta[d][1,3]-dioxol-4-yl]oxy]-1-ethanol (0.60 g, prepared according to the example 8) in tetrahydrofuran (5 ml) was added N,N-diisopropylethyl amine (0.713 g) while maintaining the temperature at about 20-25° C. A solution of 6-chloro-4-[[N-[(1R,2S)-2-(3,4-difluorophenyl)cyclopropan-1-yl]-N-tert-butoxycarbonyl]amino]-5-nitro-2-(propylthio)pyrimidine (1.38 g) in tetrahydrofuran (8 ml) was to the above solution while maintaining the temperature at about 20-25° C. over a period of 10-15 minutes, followed by stirring for 2 hours at the same temperature. After completion of the reaction, toluene (10 ml) and saturated sodium chloride solution (10 ml) were added to the reaction mass. The resulting mass was stirred for 5 minutes, followed by layer separation. The aqueous layer was extracted twice with toluene (2×10 ml). The combined organic layer was washed with saturated sodium chloride solution (10 ml) and then dried over sodium sulfate. The organic layer containing the product was evaporated to dryness under reduced pressure. The concentrated mass was further purified (silica gel, 25% ethyl acetate in hexane) to produce 0.67 g of 2-[[(3 aR,4S,6R,6aS)-6-[[4-[N-[(1R,2S)-2-(3,4-difluorophenyl)cyclopropan-1-yl]-N-tert-butoxy carbonyl]amino]-2-(propylthio)-5-nitro pyrimidin-6-yl]-2,2-dimethyl-tetrahydro-3aH-cyclopenta[d][1,3]dioxol-4-yl]oxy]ethanol.

Mass [M−H]: 680.3.

### Step-4: Preparation of 2-[[(3aR,4S,6R,6aS)-6-[[4-[N-[(1R,2S)-2-(3,4-difluorophenyl)cyclopropan-1-yl]-N-tert-butoxycarbonyl]amino]-2-(propylthio)-5-aminopyrimidin-6-yl]-2,2-dimethyl-tetrahydro-3aH-cyclopenta[d][1,3]dioxol-4-yl]oxy]ethanol

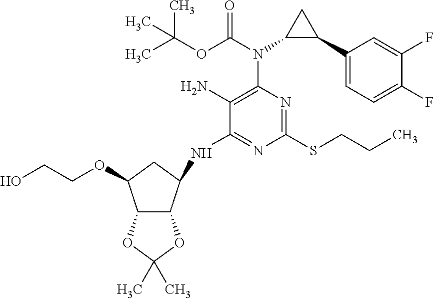 [SMILES: CCCSc1nc(N[C@@H]2C[C@H](OCCO)[C@H]3OC(C)(C)O[C@H]32)c(N)c(N(C(=O)OC(C)(C)C)[C@@H]2C[C@H]2c2ccc(F)c(F)c2)n1]

A solution of sodium dithionite (1 g) in water (2 ml) was added to a mixture of a solution of 2-[[(3aR,4S,6R,6aS)-6-[[4-[N-[(1R,2S)-2-(3,4-difluorophenyl)cyclopropan-1-yl]-N-tert-butoxycarbonyl]amino]-2-(propylthio)-5-nitropyrimidin-6-yl]-2,2-dimethyl-tetrahydro-3aH-cyclopenta[d][1,3]dioxol-4-yl]oxy]ethanol (0.10 g) in acetone (10 ml) and a solution of sodium bicarbonate (0.45 g) in water (5 ml), while maintaining the temperature at about 20-25° C., followed by stirring for 2 hours at the same temperature. After completion of the reaction, toluene (10 ml) and water (10 ml) were added to the reaction mass. The resulting reaction mass was stirred for 5 minutes, followed by layer separation. The aqueous layer was extracted with toluene (10 ml). The combined organic layer containing the product was washed with a saturated sodium chloride solution (10 ml) and dried over sodium sulfate. The organic layer containing the product was evaporated to dryness under reduced pressure to produce 0.07 g of 2-[[(3aR,4S,6R,6aS)-6-[[4-[N-[(1R,2S)-2-(3,4-difluorophenyl)cyclopropan-1-yl]-N-tert-butoxycarbonyl]amino]-2-(propylthio)-5-aminopyrimidin-6-yl]-2,2-dimethyl-tetrahydro-3aH-cyclopenta[d][1,3]dioxol-4-yl]oxy]ethanol.

Mass [M+H]: 652.3.

### Step-5: Preparation of 2-[[(3aR,4S,6R,6aS)-6-[7-[[[N-(1R,2S)-2-(3,4-difluorophenyl)-cyclopropan-1-yl]-N-tert-butoxycarbonyl]amino]-5-(propylthio)-3H-[1,2,3]triazolo[4,5-d]pyrimidin-3-yl]-2,2-dimethyl-tetrahydro-3aH-cyclopenta[d][1,3]dioxol-4-yl]oxy]ethanol

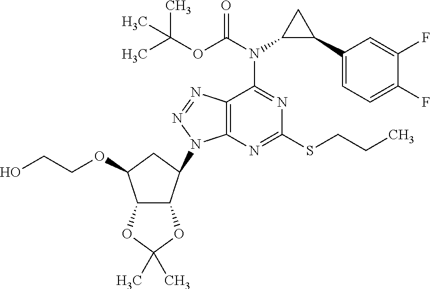 [SMILES: CCCSc1nc(N(C(=O)OC(C)(C)C)[C@@H]2C[C@H]2c2ccc(F)c(F)c2)c2nnn([C@@H]3C[C@H](OCCO)[C@H]4OC(C)(C)O[C@H]43)c2n1]

Acetic acid (1.54 g) was added to a mixture of 2-[[(3aR,4S,6R,6aS)-6-[[4-[N-[(1R,2S)-2-(3,4-difluorophenyl)cyclopropan-1-yl]-N-tert-butoxycarbonyl]amino]-2-(propylthio)-5-amino pyrimidin-6-yl]-2,2-dimethyl-tetrahydro-3aH-cyclopenta[d][1,3]dioxol-4-yl]oxy]ethanol (2.8 g), toluene (20 ml), sodium nitrite (0.34 g) and water (2 ml) while maintaining the temperature at 5-10° C., followed by stirring the mixture for 1 hour at the same temperature. After completion of the reaction, a solution of potassium carbonate (1 g) in water (20 ml) was added to the reaction mass. The resulting mass was stirred for 5 minutes, followed by layer separation. The aqueous layer was extracted twice with toluene (2×50 ml). The combined organic layer was washed with water (50 ml). The resulting organic layer was evaporated to dryness under reduced pressure. The concentrated mass was further purified (silica gel, 25% ethyl acetate in hexane) to produce 1.6 g of 2-[[(3aR,4S,6R,6aS)-6-[7-[[[N-(1R,2S)-2-(3,4-difluorophenyl)-cyclopropan-1-yl]-N-tert-butoxycarbonyl]amino]-5-(propylthio)-3H-[1,2,3]triazolo[4,5-d]pyrimidin-3-yl]-2,2-dimethyl-tetrahydro-3aH-cyclopenta[d][1,3]dioxol-4-yl]oxy]ethanol.

Mass [M+H]: 663.4; and [M+Na]: 685.3.

### Step-6: Preparation of [1S-(1α,2α,3β(1S*,2R*),5β)]-3-[7-[2-(3,4-difluorophenyl)cyclopropyl]amino]-5-(propylthio)-3H-1,2,3-triazolo[4,5-d]pyrimidin-3-yl)-5-(2-hydroxyethoxy)-cyclopentane-1,2-diol (Ticagrelor)

A mixture of 2-[[(3 aR,4 S,6R,6aS)-6-[7-[[[N-(1R,2 S)-2-(3,4-difluorophenyl)-cyclopropan-1-yl]-N-tert-butoxycarbonyl]amino]-5-(propylthio)-3H-[1,2,3]triazolo[4,5-d]pyrimidin-3-yl]-2,2-dimethyl-tetrahydro-3aH-cyclopenta[d][1,3]dioxol-4-yl]oxy]ethanol (0.5 g), methanol (1.5 ml), concentrated hydrochloric acid (1.2 ml) and toluene (2.5 ml) was stirred for 2 hours while maintaining the temperature at about 25-30° C. After completion of the reaction, toluene (5 ml) and water (5 ml) were added to the reaction mass, followed by the layer separation and washing the aqueous layer containing product with toluene (10 ml). To the aqueous layer was added a solution of sodium carbonate in water to adjust the pH to more than 8, followed by extracting twice with ethyl acetate (2×15 ml). The combined organic layer containing the product was washed with saturated sodium chloride (10 ml). The resulting organic layer was dried over sodium sulfate and then evaporated to dryness under reduced pressure to produce 0.3 g of ticagrelor.

Mass [M−H]: 521.2.

### Example 11

### Preparation of [1S-[1a,2a,3b(1S*,2R*),5b]]-3-[7-[2-(3,4-difluorophenyl)-cyclopropylamino]-5-(propylthio)-3H-1,2,3-triazolo[4,5-d]pyrimidin-3-yl]-5-(2-hydroxy ethoxy)cyclopentane-1,2-diol (Ticagrelor)

### Step 1: Preparation of 2-({(3aR,4S,6R,6aS)-6-[7-{[[N-(1R,2S)-2-(3,4-difluorophenyl)-cyclopropylamino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol

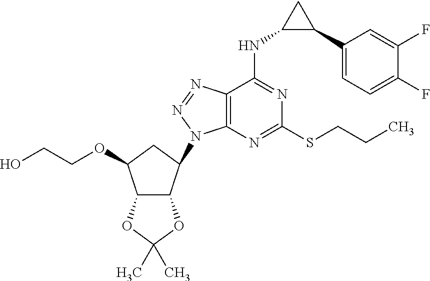 [SMILES: CCCSc1nc(N[C@@H]2C[C@H]2c2ccc(F)c(F)c2)c2nnn([C@@H]3C[C@H](OCCO)[C@H]4OC(C)(C)O[C@H]43)c2n1]

To a solution of 2-({(3aR,4S,6R,6aS)-6-[7-{[[N-(1R,2S)-2-(3,4-difluorophenyl)-cyclopropan-1-yl]-N-tert-butoxycarbonyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol (5 gm) in acetone (50 ml) was added iodine crystal (2 gm) at 25 to 30° C. The resulting solution was heated to 55 to 60° C. with continuous stirring for 2 hrs. After completion of the reaction, the reaction mixture was cooled to 40° C., followed by distillation of acetone under vacuum below 40° C. The residue was cooled to 25-30° C. and water (50 ml) and dichloromethane (50 ml) were added at 25-30° C. followed by addition of sodium thiosulphate (10 gm). The resulting solution was stirred for 30 minutes followed by layer separation. The organic layer was washed with water (50 ml). The organic layer was distilled out under vacuum below 40° C. and degassed to provide 2-({(3aR,4S,6R,6aS)-6-[7-{[[N-(1R,2S)-2-(3,4-difluorophenyl)-cyclopropylamino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol (4 gm).

### Step 2: Preparation of 2-({(3aR,4S,6R,6aS)-6-[7-{[N-(1R,2S)-2-(3,4-difluorophenyl)-cyclopropan-1-yl]-N-benzyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol

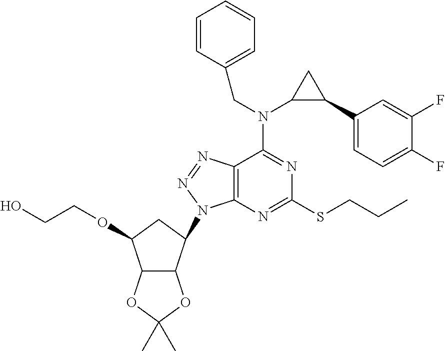 [SMILES: CCCSc1nc(N(Cc2ccccc2)C2C[C@H]2c2ccc(F)c(F)c2)c2nnn([C@@H]3C[C@H](OCCO)C4OC(C)(C)OC43)c2n1]

Benzyl bromide (1.568 gm) and powdered potassium carbonate (4.912 gm) were added to a solution of 2-({(3aR,4S,6R,6aS)-6-[7-{[[N-(1R,2S)-2-(3,4-difluorophenyl)-cyclopropylamino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol (4 gm) in acetone (120 ml). The reaction mixture was heated to 55 to 60° C. and continuously stirred for 20 hours at the same temperature. After completion of the reaction, the acetone was distilled out under vacuum and the residue was cooled to 25-30° C. Water (40 ml) and dichloromethane (40 ml) were added in to solution and stirred for 15 minutes at 25-30° C. The layers were separated and the organic layer was distilled under vacuum at 40° C. and degassed to get 2-({(3aR,4S,6R,6aS)-6-[7-{[[N-(1R,2S)-2-(3,4-difluorophenyl)-cyclopropan-1-yl]-N-benzyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol (5.5 gm).

### Step 3: Preparation of [1S-[1a,2a,3b(1S*,2R*),5b]]-3-[7-[2-(3,4-difluorophenyl)-cyclopropyl-1 yl]-N-benzyl]amino]-5-(propylthio)-3H-1,2,3-triazolo[4,5-d]pyrimidin-3-yl]-5-(2-hydroxy ethoxy)cyclopentane-1,2-diol

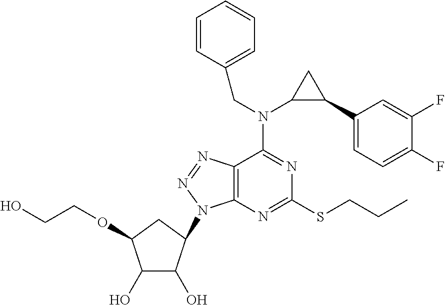 [SMILES: CCCSc1nc(N(Cc2ccccc2)C2C[C@H]2c2ccc(F)c(F)c2)c2nnn([C@@H]3C[C@H](OCCO)C(O)C3O)c2n1]

To the solution of 2-({(3aR,4S,6R,6aS)-6-[7-{[[N-(1R,2S)-2-(3,4-difluorophenyl)-cyclopropan-1-yl]-N-benzyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol (5.5 gm) and methanol (40 ml) at 20-25° C., conc. hydrochloric acid (13.5 ml) was added at 20-25° C. in 30 minutes followed by stirring the reaction mixture at 20-25° C. for 5 hrs. After the completion of reaction, the reaction mixture was washed with toluene (2×20 ml) at 25-30° C. and aqueous potassium carbonate was added and pH was adjusted to 10 at 25-30° C. The resulting solution was extracted with dichloromethane (50 ml) and organic layer was washed with water (50 ml). The layers were separated and the organic layer was distilled under vacuum below 40° C. and degassed. The residue was dissolved in 35 ml of isopropyl alcohol at 55 to 60° C., further cooled to 25 to 30° C. and maintained for 2 hours followed by drying at 50-55° C. to provide pure [1S-[1a,2a,3b(1S*,2R*),5b]]-3-[7-[2-(3,4-difluorophenyl)-cyclopropyl-1yl]-N-benzyl]amino]-5-(propylthio)-3H-1,2,3-triazolo[4,5-d]pyrimidin-3-yl]-5-(2-hydroxy ethoxy)cyclopentane-1,2-diol (5 gm), HPLC purity—99.85%.

### Step 4: Preparation of [1S-[1a,2a,3b(1S*,2R*),5b]]-3-[7-[2-(3,4-difluorophenyl)-cyclopropylamino]-5-(propylthio)-3H-1,2,3-triazolo[4,5-d]pyrimidin-3-yl]-5-(2-hydroxy ethoxy)cyclopentane-1,2-diol (Ticagrelor)

10% palladium on carbon and formic acid in ethanol were added to [1S-[1a,2a,3b (1S*,2R*),5b]]-3-[7-[2-(3,4-difluorophenyl)-cyclopropyl-1yl]-N-benzyl]amino]-5-(propylthio)-3H-1,2,3-triazolo[4,5-d]pyrimidin-3-yl]-5-(2-hydroxyethoxy)cyclopentane-1,2-diol (5 gm) at 50° C. in three portions. The catalyst was filtered through high-low bed and the ethanol was distilled out under vacuum to get [1S-[1a,2a,3b(1S*,2R*),5b]]-3-[7-[2-(3,4-difluorophenyl)-cyclopropylamino]-5-(propylthio)-3H-1,2,3-triazolo[4,5-d]pyrimidin-3-yl]-5-(2-hydroxy ethoxy)cyclopentane-1,2-diol (4 gm).

HPLC purity—99.7%

### Example 12

### Preparation of [1S-[1a,2a,3b(1S*,2R*),5b]]-3-[7-[2-(3,4-difluorophenyl)-cyclopropylamino]-5-(propylthio)-3H-1,2,3-triazolo[4,5-d]pyrimidin-3-yl]-5-(2-hydroxy ethoxy)cyclopentane-1,2-diol (Ticagrelor)

### Step 1: Preparation of 2-({(3 aR,4S,6R,6aS)-6-[7-{[[N-(1R,2S)-2-(3,4-difluorophenyl)-cyclopropan-1-yl]-N-tertbutoxycarbonyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)-O-tert-butoxycarbonylethanol

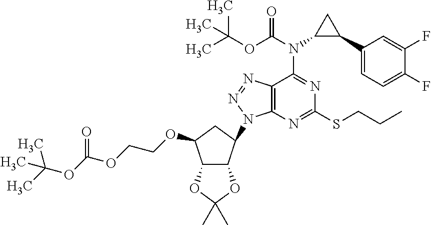 [SMILES: CCCSc1nc(N(C(=O)OC(C)(C)C)[C@@H]2C[C@H]2c2ccc(F)c(F)c2)c2nnn([C@@H]3C[C@H](OCCOC(=O)OC(C)(C)C)[C@H]4OC(C)(C)O[C@H]43)c2n1]

To the solution of 2-({(3aR,4S,6R,6aS)-6-[7-{[[N-(1R,2S)-2-(3,4-difluorophenyl)-cyclopropan-1-yl]-N-tert-butoxycarbonyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)ethanol (6.5 gm) in acetone (100 ml) maintained at 25-30° C., potassium carbonate (3.38 gm) and di-tert-butyl dicarbonate (6.42 gm) were added. The reaction mixture was heated to 55 to 60° C. and stirred for 24 hrs. After the completion of the reaction, acetone was distilled out under vacuum and the residue was cooled at 25-30° C. Water (50 ml) and dichloromethane (50 ml) were added and resulting mixture was stirred for 15 minutes. The layers were separated and the organic layer was washed with water (50 ml) at 25-30° C. The dichloromethane was distilled out under vacuum at 40° C. and degassed for 30 minutes at 40° C. The residue was dissolved isopropyl alcohol (45 ml) at 55 to 60° C., further cooled to 25 to 30° C. and maintained for 2 hours followed by drying at 50-55° C. to provide pure 2-({(3aR,4S,6R,6aS)-6-[7-{[[N-(1R,2S)-2-(3,4-difluorophenyl)-cyclopropan-1-yl]-N-tertbutoxycarbonyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)-O-tert-butoxycarbonylethanol (6.5 gm), HPLC purity-99.9%.

### Step 2: Preparation of [1S-[1a,2a,3b(1S*,2R*),5b]]-3-[7-[2-(3,4-difluorophenyl)-cyclopropylamino]-5-(propylthio)-3H-1,2,3-triazolo[4,5-d]pyrimidin-3-yl]-5-(2-hydroxy ethoxy)cyclopentane-1,2-diol (Ticagrelor)

To the solution of 2-({(3aR,4S,6R,6aS)-6-[7-{[[N-(1R,2S)-2-(3,4-difluorophenyl)-cyclopropan-1-yl]-N-tertbutoxycarbonyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)-O-tert-butoxycarbonylethanol (6 gm) in methanol (40 ml) at 20-25° C. concentrated hydrochloric acid (15 ml) was added in 30 minutes maintaining the temperature at 20-25° C. The reaction mixture was heated at 50° C. and stirred at 50-55° C. for 5 hours. After the completion of the reaction, the reaction mixture was cooled to 25-30° C. and washed with toluene (2×20 ml) at 25-30° C. Aqueous potassium carbonate was added to resulting solution and pH was adjusted to 10 at 25-30° C. The reaction mass was extracted with dichloromethane (50 ml) and the organic layer was washed with water (50 ml). The dichloromethane layer containing the product was distilled out under vacuum below 40° C. and degassed to provide [1S-[1a,2a,3b(1S*,2R*),5b]]-3-[7-[2-(3,4-difluorophenyl)-cyclopropylamino]-5-(propylthio)-3H-1,2,3-triazolo[4,5-d]pyrimidin-3-yl]-5-(2-hydroxy ethoxy)cyclopentane-1,2-diol (4 gm).

HPLC purity—99.7%.

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

The term “micronization” used herein means a process or method by which the size of a population of particles is reduced.

As used herein, the term “micron” or “μm” both are equivalent refers to “micrometer” which is 1×10-6 meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent.

The important characteristics of the PSD are the (D90), which is the size, in microns, below which 90% of the particles by volume are found, and the (D50), which is the size, in microns, below which 50% of the particles by volume are found. Thus, a D90 or d(0.9) of less than 300 microns means that 90 volume-percent of the particles in a composition have a diameter less than 300 microns.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A process for preparing a triazolo[4,5-d]pyrimidine compound of formula I: 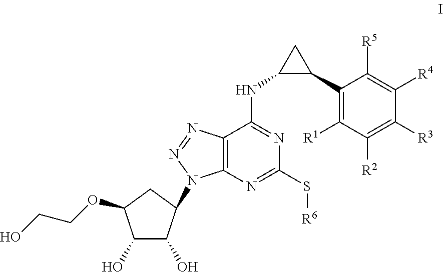
or a pharmaceutically acceptable salt thereof; wherein R¹, R², R³, R⁴ and R⁵ are, each independently, selected from hydrogen and a halogen atom, wherein the halogen atom is F, Cl, Br or I; and R⁶ is C₁₋₆ alkyl; comprising:
a) reacting a substituted phenylcyclopropylamine compound of formula II: 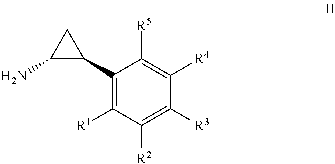
or an acid addition salt thereof, wherein R¹, R², R³, R⁴ and R⁵ are as defined in formula I, with a compound of formula III: 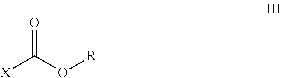
wherein ‘X’ is a leaving group selected from a halogen atom, —OC(O)OR⁷ and C₁₋₄ alkoxy, wherein R⁷ is C₁₋₄ alkyl; R is C₁₋₆ alkyl or benzyl, wherein the phenyl ring of benzyl is optionally substituted by halogen, nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃;
in the presence of a first base in a first solvent to produce a carbamic acid ester compound of formula IV: 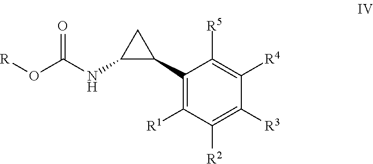
or an acid addition salt thereof, wherein R, R¹, R², R³, R⁴ and R⁵ are as defined above;
b) reacting the carbamic acid ester compound of formula IV with a dichloropyrimidine compound of formula V: 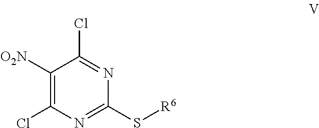
wherein R⁶ is C₁₋₆ alkyl; in the presence of a second base in a second solvent to produce a pyrimidine compound of formula VI: 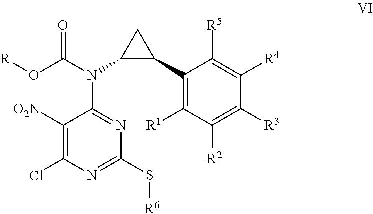
wherein R, R¹, R², R³, R⁴, R⁵ and R⁶ are as defined above;
c) reacting the compound formula VI with a cyclopentanamine compound formula VII; 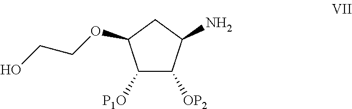
or an acid addition salt thereof, wherein P₁ and P₂ are protecting groups, or P₁ and P₂ together with the atoms to which they are attached form an alkylidene ring, wherein the alkylidene ring is methylidene or isopropylidene ring;
in the presence of a third base in a third solvent to produce a diaminopyrimidine compound of formula VIII: 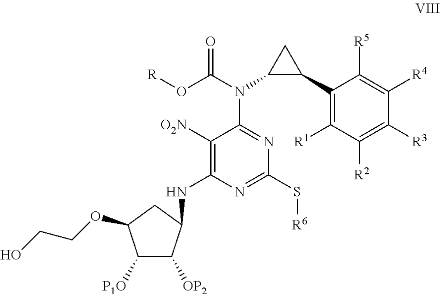
or an acid addition salt thereof, wherein P₁, P₂, R, R¹, R², R³, R⁴, K R⁵ and R⁶ are as defined above;
d) reducing the diaminopyrimidine compound formula VIII using a reducing agent in a fourth solvent to produce a triaminopyrimidine compound of formula IX: 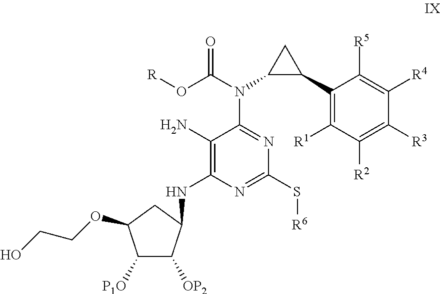
or an acid addition salt thereof, wherein P₁, P₂, R, R¹, R², R³, R⁴, R⁵ and R⁶ are as defined above;
e) reacting the triaminopyrimidine compound of formula IX with a nitrite reagent in a fifth solvent in the presence of an acid to produce a triazol compound of formula X: 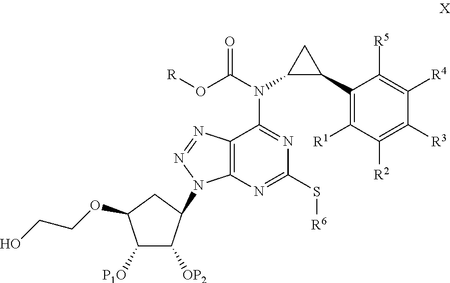
wherein P₁, P₂, R, R¹, R², R³, R⁴, R⁵ and R⁶ are as defined above; and
f) subjecting the triazol compound of formula X to acid hydrolysis or hydrogenolysis with a suitable acid in a sixth solvent to produce the triazolo[4,5-d]pyrimidine compound of formula I, and optionally converting the compound of formula I obtained into a pharmaceutically acceptable salt thereof.

2. The process of claim 1, wherein the compounds of formulae I, II, III, IV, V, VI, VIII, IX, X are defined according to one of (a), (b), or (c):
(a) the halogen atom as defined in the compounds of formulae I, II, IV, VI, VIII, IX and X is F or Cl; wherein the group ‘R⁶’ in the compounds of formulae I, V, VI, VIII, IX and X is selected from methyl, ethyl, n-propyl, iso-propyl, n-butyl, iso-butyl and sec.-butyl; wherein the halogen atom in the compounds of formula III is F, Cl, Br or I; wherein the group ‘R’ in the compounds of formula III is selected from methyl, ethyl, n-propyl, iso-propyl, n-butyl, iso-butyl, tert-butyl and sec.-butyl; and wherein the group ‘R⁷’ in the —OC(O)OR⁷ as defined for the formula III is selected from methyl, ethyl, n-propyl, iso-propyl, n-butyl, iso-butyl, tert-butyl and sec.-butyl;
(b) the halogen atom as defined in the compounds of formulae I, II, IV, VI, VIII, IX and X is F; wherein the group ‘R⁶’ in the compounds of formulae I, V, VI, VIII, IX and X is n-propyl; wherein the halogen atom in the compound of formula III is Cl; wherein the group ‘R’ in the compounds of formula III is tert-butyl; and wherein the group ‘R⁷’ in the —OC(O)OR⁷ as defined for the formula III is tert-butyl;
(c) the triazolo[4,5-d]pyrimidine derivative of formula I obtained is ticagrelor, [1S-(1α,2α,3β(1S*,2R*),5β)]-3-[7-[2-(3,4-difluorophenyl)cyclopropyl]amino]-5-(propylthio)-3H-1,2,3-triazolo[4,5-d]pyrimidin-3-yl)-5-(2-hydroxy ethoxy)-cyclopentane-1,2-diol, of formula Ia (formula I, wherein R¹, R² and R⁵ are H; R³ and R⁴ are F; and R⁶ is n-propyl group): 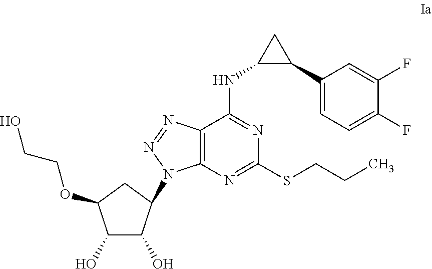 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]
or a pharmaceutically acceptable salt thereof

3. (canceled)

4. (canceled)

5. A compound selected from one of (A), (B), (C), (D), (E), (F), (G), (H), (I), (J), or (K):
(A) a carbamic acid ester compound of formula IV: 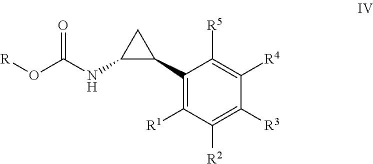
or an acid addition salt thereof, wherein R¹, R², R³, R⁴ and R⁵ are, each independently, selected from hydrogen and a halogen atom, wherein the halogen atom is F, Cl, Br or I; and R is C₁₋₆ alkyl or benzyl, wherein the phenyl ring of benzyl is optionally substituted by halogen, nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃;
(B) a pyrimidine compound of formula VI: 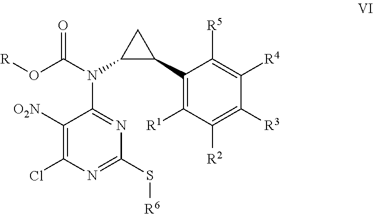
or a pharmaceutically acceptable salt thereof, wherein R¹, R², R³, R⁴ and R⁵ are, each independently, selected from hydrogen and a halogen atom, wherein the halogen atom is F, Cl, Br or I; R⁶ is C₁₋₆ alkyl; and R is C₁₋₆ alkyl or benzyl, wherein the phenyl ring of benzyl is optionally substituted by halogen, nitro, S(O)₂(C₁₋₄ alkyl), cyano, C₁₋₄ alkyl, C₁₋₄ alkoxy, C(O)(C₁₋₄ alkyl), N(C₁₋₆alkyl)₂, CF₃ or OCF₃;
(C) a diaminopyrimidine compound of formula VIII: 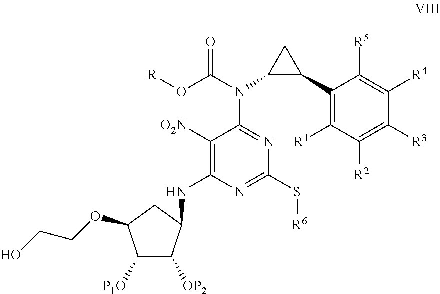
or an acid addition salt thereof, wherein R¹, R², R³, R⁴ and R⁵ are, each independently, selected from hydrogen and a halogen atom, wherein the halogen atom is F, Cl, Br or I; R⁶ is C₁₋₆ alkyl; R is C₁₋₆ alkyl or benzyl, wherein the phenyl ring of benzyl is optionally substituted by halogen, nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃; and P₁ and P₂ are protecting groups, or P₁ and P₂ together with the atoms to which they are attached form an alkylidene ring;
(D) a triaminopyrimidine compound of formula IX: 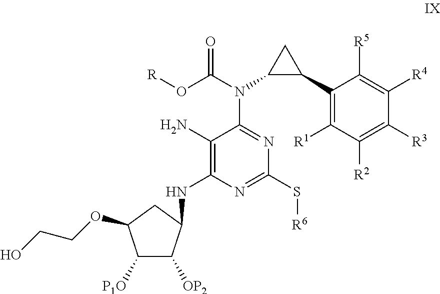
or an acid addition salt thereof, wherein R¹, R², R³, R⁴ and R⁵ are, each independently, selected from hydrogen and a halogen atom, wherein the halogen atom is F, Cl, Br or I; R⁶ is C₁₋₆ alkyl; R is C₁₋₆ alkyl or benzyl, wherein the phenyl ring of benzyl is optionally substituted by halogen, nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃; and P₁ and P₂ are protecting groups, or P₁ and P₂ together with the atoms to which they are attached form an alkylidene ring;
(E) a triazol compound of formula X: 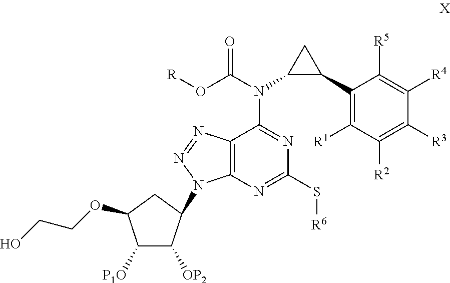
or a pharmaceutically acceptable salt thereof, wherein R¹, R², R³, R⁴ and R⁵ are, each independently, selected from hydrogen and a halogen atom, wherein the halogen atom is F, Cl, Br or I; R⁶ is C₁₋₆ alkyl; R is C₁₋₆ alkyl or benzyl, wherein the phenyl ring of benzyl is optionally substituted by halogen, nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃; and P₁ and P₂ are protecting groups, or P₁ and P₂ together with the atoms to which they are attached form an alkylidene ring;
(F) a benzyl protected compound of formula XIII: 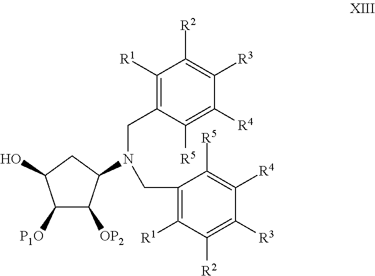
or a pharmaceutically acceptable salt thereof, wherein R¹, R², R³, R⁴ and R⁵ are, each independently, selected from hydrogen, F, Cl, Br, I, nitro, C₁-C₃-alkyl, and C₁-C₃-alkoxy substituents; and P₁ and P₂ are protecting groups, or P₁ and P₂ together with the atoms to which they are attached form an alkylidene ring;
(G) an ester compound of formula XV: 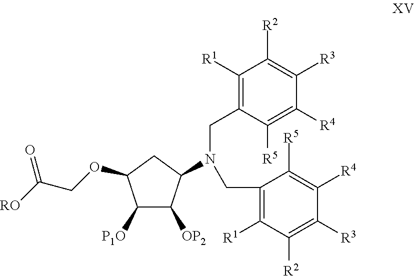
or a pharmaceutically acceptable salt thereof, wherein R¹, R², R³, R⁴ and R⁵ are, each independently, selected from hydrogen, F, Cl, Br, I, nitro, C₁-C₃-alkyl, and C₁-C₃-alkoxy substituents; R is C₁₋₆ straight or branched alkyl, or a benzyl group, wherein the phenyl ring of benzyl group is optionally substituted with one or more of the nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃; and P₁ and P₂ are protecting groups, or P₁ and P₂ together with the atoms to which they are attached form an alkylidene ring;
(H) a hydroxy compound of formula XVI: 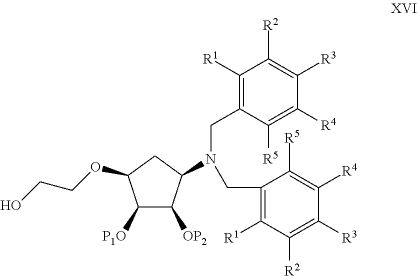
or a pharmaceutically acceptable salt thereof, wherein R¹, R², R³, R⁴ and R⁵ are, each independently, selected from hydrogen, F, Cl, Br, I, nitro, C₁-C₃-alkyl, and C₁-C₃-alkoxy substituents; and P₁ and P₂ are protecting groups, or P₁ and P₂ together with the atoms to which they are attached form an alkylidene ring;
(I) a compound of formula XVIII 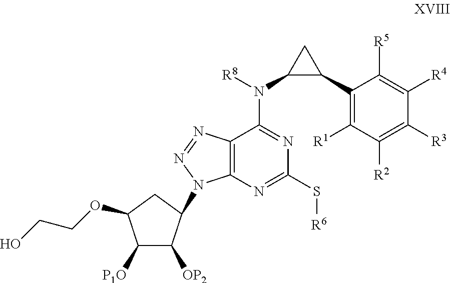
or an acid addition salt thereof, wherein R¹, R², R³, R⁴ and R⁵ are, each independently, selected from hydrogen and a halogen atom, wherein the halo en atom is F, Cl, Br or I; R⁶ is C₁₋₆ alkyl; R⁸ is selected from C₁₋₆alkyl, benzyl, substituted benzyl (C₁₋₆ alkyl)₃Si (specifically t-butyldimethylsilyl) and a C(O)C₁₋₆alkyl group and P₁ and P₂ together with the atoms to which they are attached form an isopropylidene ring;
(J) a compound of formula XIX 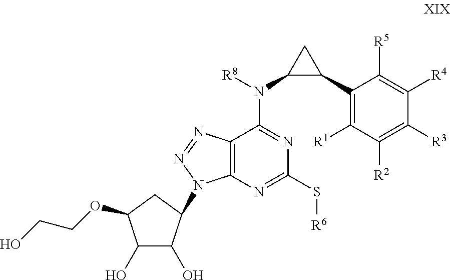
or an acid addition salt thereof, wherein R¹, R², R³, R⁴ and R⁵ are, each independently, selected from hydrogen and a halogen atom, wherein the halogen atom is F, Cl, Br or I; R⁶ is C₁₋₆ alkyl; R⁸ is selected from C₁₋₆alkyl, benzyl, substituted benzyl (C₁₋₆ alkyl)₃Si (specifically t-butyldimethylsilyl) and a C(O)C₁₋₆alkyl group, and P₁ and P₂ are hydrogen; or
(K) a compound of formula XX 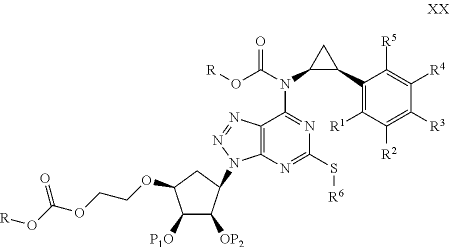
or an acid addition salt thereof, wherein R¹, R², R³, R⁴ and R⁵ are, each independently, selected from hydrogen and a halogen atom, wherein the halo en atom is F, Cl, Br or I; R⁶ is C₁₋₆ alkyl; R is C₁₋₆ alkyl or benzyl, wherein the phenyl ring of benzyl is optionally substituted by halogen, nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃; P₁ and P₂ are C₁₋₆ alkyl, benzyl, (C₁₋₆ alkyl)₃Si, and C(O)C₁₋₆ alkyl and P₁ and P₂ together with the atoms to which they are attached form an isopropylidene ring.

6. The compound of claim 5, wherein the compounds are further defined according to one of (i), (ii), (iii), (iv), (v), (vi), (vii), (viii), (ix), (x), or (xi):
(i) the carbamic acid ester compound (A) is tert-butyl [(1R,2S)-2-(3,4-difluorophenyl)cyclopropyl]carbamate of formula IVa (formula IV, wherein R¹, R² and R⁵ are H; R³ and R⁴ are F; and R is tert-butyl): 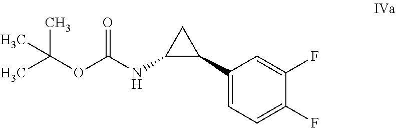 [SMILES: CC(C)(C)OC(=O)N[C@@H]1C[C@H]1c1ccc(F)c(F)c1]
or an acid addition salt thereof;
(ii) the pyrimidine compound (B) is 6-chloro-4-[[N-[(1R,2S)-2-(3,4-difluorophenyl)cyclopropan-1-yl]-N-tert-butoxycarbonyl]amino]-5-nitro-2-(propylthio)pyrimidine of formula VIa (formula VI, wherein R¹, R² and R⁵ are H; R³ and R⁴ are F; R is tert-butyl; and R⁶ is n-propyl): 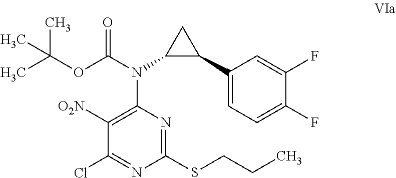 [SMILES: CCCSc1nc(Cl)c([N+](=O)[O-])c(N(C(=O)OC(C)(C)C)[C@@H]2C[C@H]2c2ccc(F)c(F)c2)n1]
or a pharmaceutically acceptable salt thereof;
(iii) the diaminopyrimidine compound (C) is 2-[[(3aR,4S,6R,6aS)-6-[4-1R,2S)-2-(3,4-difluorophenyl)cyclopropan-1-yl]-N-tert-butoxycarbonyl]amino]-2-(propylthio)-5-nitropyrimidin-6-yl]-2,2-dimethyl-tetrahydro-3aH-cyclopenta[d][1,3]dioxol-4-yl]oxy]ethanol of formula VIIIa (formula VIII, wherein R¹, R² and R⁵ are H; R³ and R⁴ are F; R is tert-butyl; R⁶ is n-propyl; and the two groups P₁ and P₂ together with the atoms to which they are attached form an isopropylidene ring): 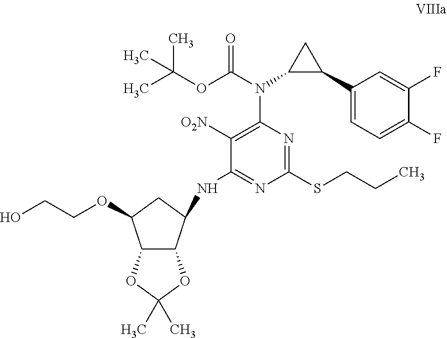 [SMILES: CCCSc1nc(N[C@@H]2C[C@H](OCCO)[C@H]3OC(C)(C)O[C@H]32)c([N+](=O)[O-])c(N(C(=O)OC(C)(C)C)[C@@H]2C[C@H]2c2ccc(F)c(F)c2)n1]
or an acid addition salt thereof;
(iv) the triaminopyrimidine compound (D) is 2-[[(3aR,4S,6R,6aS)-6-[[4-[N-[(1R,2S)-2-(3,4-difluorophenyl)cyclopropan-1-yl]-N-tert-butoxycarbonyl]amino]-2-(propylthio)-5-amino pyrimidin-6-yl]-2,2-dimethyl-tetrahydro-3aH-cyclopenta[d][1,3]dioxol-4-yl]oxy]ethanol of formula IXa (formula IX, wherein R¹, R² and R⁵ are H; R³ and R⁴ are F; R is tert-butyl; R⁶ is n-propyl; and the two groups P₁ and P₂ together with the atoms to which they are attached form an isopropylidene ring): 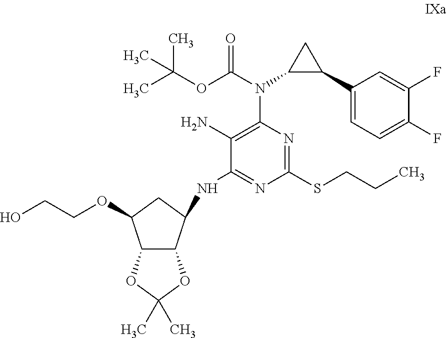 [SMILES: CCCSc1nc(N[C@@H]2C[C@H](OCCO)[C@H]3OC(C)(C)O[C@H]32)c(N)c(N(C(=O)OC(C)(C)C)[C@@H]2C[C@H]2c2ccc(F)c(F)c2)n1]
or an acid addition salt thereof; or
(v) the triazol compound (E) of formula X is 2-[[(3aR,4S,6R,6aS)-6-[7-[[[N-(1R,2S)-2-(3,4-difluorophenyl)-cyclopropan-1-yl]-N-tert-butoxycarbonyl]amino]-5-(propylthio)-3H-[1,2,3]triazolo[4,5-d]pyrimidin-3-yl]-2,2-dimethyl-tetrahydro-3aH-cyclopenta[d][1,3]dioxol-4-yl]oxy]ethanol of formula Xa (formula X, wherein R¹, R² and R⁵ are H; R³ and R⁴ are F; R is tert-butyl; R⁶ is n-propyl; and the two groups P₁ and P₂ together with the atoms to which they are attached form an isopropylidene ring): 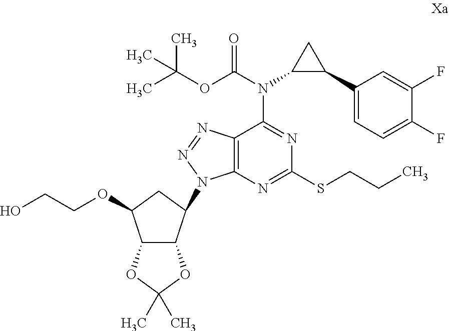 [SMILES: CCCSc1nc(N(C(=O)OC(C)(C)C)[C@@H]2C[C@H]2c2ccc(F)c(F)c2)c2nnn([C@@H]3C[C@H](OCCO)[C@H]4OC(C)(C)O[C@H]43)c2n1]
or a pharmaceutically acceptable salt thereof;
(vi) the benzyl protected compound (F) is (3aR,4S,6R,6aS)-6-(N,N-dibenzylamino)-2,2-dimethyltetrahydro-3aH-cyclopenta[d][1,3]dioxol-4-ol of formula XIII a (formula XIII, wherein R¹, R², R³, R⁴ and R⁵ are H; and the two groups P₁ and P₂ together with the atoms to which they are attached form an isopropylidene ring): 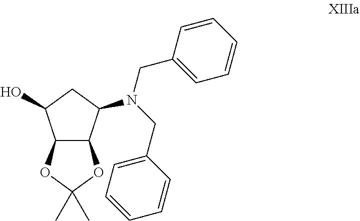 [SMILES: CC1(C)O[C@@H]2[C@H](O1)[C@H](N(Cc1ccccc1)Cc1ccccc1)C[C@@H]2O]
or a pharmaceutically acceptable salt thereof;
(vii) the ester compound (G) is tert-butyl [[(3aR,4S,6R,6aS)-6-(N,N-Dibenzylamino)-2,2-dimethyltetrahydro-3aH-cyclopenta[d][1,3]dioxol-4-yl]oxy]acetate of formula XVa (formula XV, wherein R¹, R², R³, R⁴ and R⁵ are H; R is tert-butyl; and the two groups P₁ and P₂ together with the atoms to which they are attached form an isopropylidene ring): 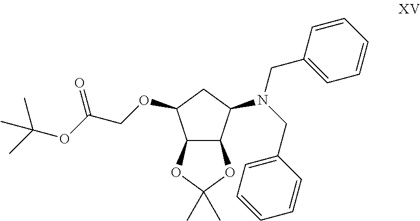 [SMILES: CC(C)(C)OC(=O)CO[C@H]1C[C@@H](N(Cc2ccccc2)Cc2ccccc2)[C@H]2OC(C)(C)O[C@H]21]
or a pharmaceutically acceptable salt thereof;
(viii) the hydroxy compound (H) is 2-[[(3aR,4S,6R,6aS)-6-(N,N-dibenzylamino)-2,2-dimethyltetrahydro-3aH-cyclopenta[d][1,3]dioxol-4-yl]oxy]ethanol of formula XVIa formula XVI, wherein R¹, R², R³, R⁴ and R⁵ are H; and the two groups P₁ and P₂ together with the atoms to which they are attached form an isopropylidene ring): 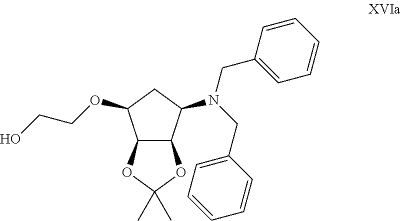 [SMILES: CC1(C)O[C@@H]2[C@H](O1)[C@H](N(Cc1ccccc1)Cc1ccccc1)C[C@@H]2OCCO]
or a pharmaceutically acceptable salt thereof;
(ix) compound (I) is 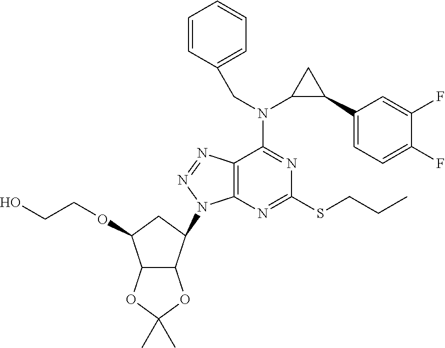 [SMILES: CCCSc1nc(N(Cc2ccccc2)C2C[C@H]2c2ccc(F)c(F)c2)c2nnn([C@@H]3C[C@H](OCCO)C4OC(C)(C)OC43)c2n1]
2-[[(3aR,4S,6R,6 as)-6-[7-[[[N-(1R,2S)-2-(3,4-difluorophenyl)-cyclopropylamino]-5-(propylthio)-3H-[1,2,3]triazolo[4,5-d]pyrimidin-3-yl]-2,2-dimethyl-tetrahydro-3aH-cyclopenta[d][1,3]dioxol-4-yl]oxy)ethanol of formula XVIII a (formula XVIII, wherein R¹, R² and R⁵ are H; R³ and R⁴ are F; R⁸ is a N-benzyl; R⁶ is n-propyl; and the two groups P₁ and P₂ together with the atoms to which they are attached form an isopropylidene ring);
(x) compound (J) of formula XIX is 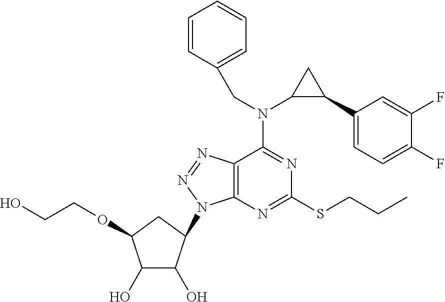 [SMILES: CCCSc1nc(N(Cc2ccccc2)C2C[C@H]2c2ccc(F)c(F)c2)c2nnn([C@@H]3C[C@H](OCCO)C(O)C3O)c2n1]
2-[[(3aR,4S,6R,6 as)-6-[7-[[[N-(1R,2S)-2-(3,4-difluorophenyl)-cyclopropylamino]-5-(propylthio)-3H-[1,2,3]triazolo[4,5-d]pyrimidin-3-yl]-5-(2-hydroxy ethoxy)cyclopentane-1,2-diol of formula XIX (formula XIX, wherein R¹, R² and R⁵ are H; R³ and R⁴ are F; R⁸ is benzyl; R⁶ is n-propyl; and the two groups P₁ and P₂ are independently H); or
(xi) the compound (K) of formula XX is 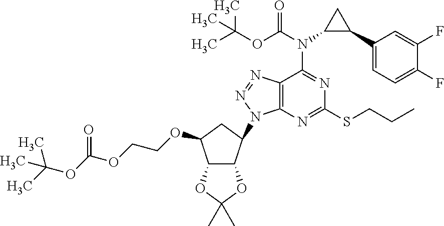 [SMILES: CCCSc1nc(N(C(=O)OC(C)(C)C)[C@@H]2C[C@H]2c2ccc(F)c(F)c2)c2nnn([C@@H]3C[C@H](OCCOC(=O)OC(C)(C)C)[C@H]4OC(C)(C)O[C@H]43)c2n1]
2-({(3 aR,4S,6R,6aS)-6-[7-{[[N-(1R,2S)-2-(3,4-difluorophenyl)-cyclopropan-1-yl]-N-tertbutoxycarbonyl]amino}-5-(propylthio)-3H-[1,2,3]triazolo[4,5-d]pyrimidin-3-yl]-2,2-dimethyl-tetrahydro-3aH-cyclopenta[d][1,3]dioxol-4-yl}oxy)-O-tert-butoxycarbonyl ethanol of formula XX a (formula XX, wherein R¹, R² and R⁵ are H; R³ and R⁴ are F; R is tertbutyl; R6 is n-propyl; and the two groups P₁ and P₂ are independently H).

7-14. (canceled)

15. The process of claim 1, wherein the protecting groups P₁ and P₂ are defined according to one of (a), (b), or (c):
(a) the protecting groups P₁ and P₂ in the compounds of formulae VII, VIII, IX and X are C₁₋₆ alkyl, benzyl, (C₁₋₆ alkyl)₃Si, and C(O)C₁₋₆ alkyl,
(b) the protecting groups in the compounds of formulae VII, VIII, IX and X are methyl, benzyl, t-butyldimethylsilyl and acetyl; or
(c) the protecting groups P₁ and P₂ together with the atoms to which they are attached form an isopropylidene ring

16. (canceled)

17. (canceled)

18. The process of claim 1, wherein the solvents are defined according to (a) or (b):
(a) the first solvent used in step-(a) is selected from the group consisting of a ketone, an aliphatic or alicyclic hydrocarbon, a chlorinated aliphatic or aromatic hydrocarbon, an aromatic mono or dinitro hydrocarbon, an aliphatic or cyclic ether, a polar aprotic solvent, and mixtures thereof; wherein the second solvent used in step-(b) is selected from the group consisting of acetone, methyl ethyl ketone, methyl isobutyl ketone, methyl tert-butyl ketone, acetonitrile, tetrahydrofuran, 2-methyl tetrahydrofuran, 1,4-dioxane, diethyl ether, diisopropyl ether, methyl tert-butyl ether, monoglyme, diglyme, n-pentane, n-hexane, n-heptane, cyclohexane, toluene, xylene, N,N-dimethylformamide, N,N-dimethylacetamide, dimethylsulfoxide, N-methylpyrrolidone, and mixtures thereof; wherein the third solvent used in step-(c) is selected from the group consisting of a ketone, an aliphatic or alicyclic hydrocarbon, a chlorinated aliphatic or aromatic hydrocarbon, an aromatic mono or dinitro hydrocarbon, an aliphatic or cyclic ether, a polar aprotic solvent, and mixtures thereof; wherein the fourth solvent used in step-(d) is selected from the group consisting of water, a ketone, an alcohol, a hydrocarbon, a cyclic ether, an aliphatic ether, a chlorinated hydrocarbon, and mixtures thereof; wherein the fifth solvent used in step-(e) is selected from the group consisting of water, a hydrocarbon, cyclic ethers, an ether, an ester, a nitrile, an aliphatic amide, a chlorinated hydrocarbon, and mixtures thereof; and wherein the sixth solvent used in step-(f) is selected from the group consisting of an alcohol, a hydrocarbon, a cyclic ether, an aliphatic ether, a chlorinated hydrocarbon, and mixtures thereof; or
(b) the first solvent is dichloromethane; wherein the second solvent is tetrahydrofuran; wherein the third solvent is tetrahydrofuran; wherein the fourth solvent is selected from the group consisting of water, acetone, tetrahydrofuran, and mixtures thereof; wherein the fifth solvent is selected from the group consisting of toluene, water, dichloromethane, 2-methyl tetrahydrofuran, tetrahydrofuran, and mixtures thereof; and wherein the sixth solvent used in step-(f) is selected from the group consisting of toluene, dichloromethane, 2-methyl tetrahydrofuran, methanol, isopropyl alcohol, tetrahydrofuran, and mixtures thereof.

19. (canceled)

20. The process of claim 1, wherein the process is further defined according to one of (a), (b), (c), or (d):
(a) the compound of formula III used in step-(a) is selected from the group consisting of di-alkyldicarbonates, alkyl chloroformates, substituted aryl dicarbonates and chloroformates; wherein the reducing agent used in step-(d) is selected from the group consisting of ferric chloride-hydrazine hydrate, sodium dithionite, tin chloride hydrate, tin chloride hydrate-hydrochloric acid, tin-hydrochloric acid, zinc-ammonium formate, zinc-formic acid, zinc-acetic acid, zinc-hydrochloric acid, zinc-hydrazinium monoformate, magnesium-ammonium formate, zinc dust-ammonium chloride, palladium, platinum, raney-nickel, ferrous sulfate heptahydrate in aqueous ammonia, iron, zinc, cobalt, and mixtures thereof; wherein the nitrite reagent used in step-(e) is a metal nitrite or an alkyl nitrite; wherein the acid used in step-(e) is a mineral acid or an organic acid; and wherein the acid used in step-(f) is a mineral acid or an organic acid;
(b) the compound of formula III used in step-(a) is di-tert-butyldicarbonate; wherein the reducing agent used in step-(d) is sodium dithionite; wherein the nitrite reagent is selected from the group consisting of sodium nitrite, potassium nitrite, lithium nitrite, butyl nitrite, isoamyl nitrite, and mixtures thereof; wherein the acid used in step-(e) is selected from the group consisting of hydrochloric acid, hydrobromic acid, sulfuric acid, acetic acid, propionic acid, butanoic acid, pivalic acid, pentanoic acid, hexanoic acid, methane sulfonic acid, p-toluene sulfonic acid, and mixtures thereof; and wherein the acid used in step-(e) is selected from the group consisting of hydrochloric acid, hydrobromic acid, sulfuric acid, acetic acid, propionic acid, butanoic acid, pivalic acid, pentanoic acid, hexanoic acid, methane sulfonic acid, p-toluene sulfonic acid, camphor sulphonic acid and mixtures thereof;
(c) the reduction in step-(d) is carried out in the presence or absence of hydrogen gas; or,
(d) the reduction in step-(d) is carried out by catalytic hydrogen transfer process employing a catalytic transfer hydrogenation 1,4-cyclohexadiene, cyclohexene, ammonium formate, formic acid, sodium formate, hydrazine, 1,3-cyclohexadiene, trialkylammonium formates, and mixtures thereof.

21-23. (canceled)

24. A process for the preparation of a substituted cyclopentanamine derivative of formula VII: 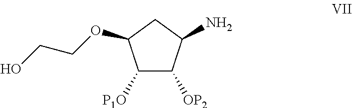
or an acid addition salt thereof; wherein P₁ and P₂ both represents hydrogen or a protecting group, or P₁ and P₂ together with the atoms to which they are attached form an alkylidene ring such as a methylidene or isopropylidene ring; comprising:
a) reacting a cyclopentanol compound of formula XI: 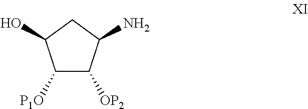
or an acid addition salt thereof, wherein P₁ and P₂ are as defined above, with an alkylating agent of formula XII: 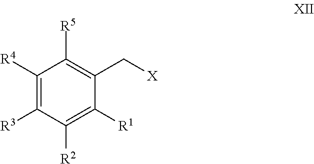
wherein ‘X’ is a leaving group, selected from the group consisting of mesyl, tosyl, Cl, Br and I; and wherein R¹, R², R³, R⁴ and R⁵ are, each independently, selected from hydrogen, F, Cl, Br, I, nitro, C₁-C₃-alkyl, and C₁-C₃-alkoxy substituents; in the presence of a base in a first solvent to produce a benzyl protected compound of formula XIII: 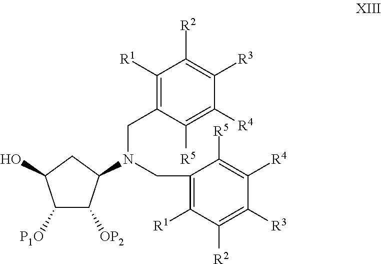
wherein P₁, P₂, R¹, R², R³, R⁴ and R⁵ are as defined above;
b) reacting the compound of formula XIII with a compound of formula XIV: 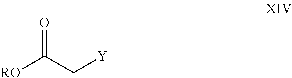
wherein ‘Y’ is a leaving group, selected from the group consisting of mesyl, tosyl, Cl, Br and I; R is C₁₋₆ straight or branched alkyl, or a benzyl group, wherein the phenyl ring of benzyl group is optionally substituted with one or more of the nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃;
in the presence of an organic or inorganic base in a second solvent to produce an ester compound of formula XV: 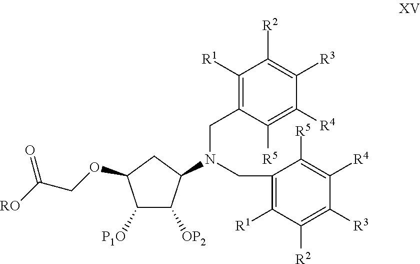
wherein P₁, P₂, R, R¹, R², R³, R⁴ and R⁵ are as defined above;
c) reducing the ester compound of formula XVI with a reducing agent in the presence of a third solvent to produce a hydroxy compound of formula XVI: 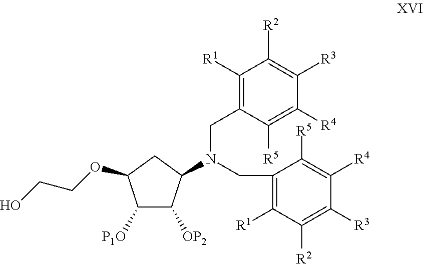
wherein P₁, P₂, R¹, R², R³, R⁴ and R⁵ are as defined above; and
d) deprotecting the compound of formula XVI in a fourth solvent to produce the substituted cyclopentanamine derivative of formula VII, and optionally converting the compound of formula VII obtained into an acid addition salt thereof.

25. The process of claim 24, wherein the compounds are further defined according to one of (a), (b), (c), or (d):
(a) the protecting groups P₁ and P₂ in the compounds of formulae VII, XI, XIII, XV and XVI are C₁₋₆ alkyl, benzyl, (C₁₋₆ alkyl)₃Si, and C(O)C₁₋₆ alkyl; wherein the leaving group ‘X’ in the compounds of formula XII is Cl or Br; wherein the groups R¹, R², R³, R⁴ and R⁵ in the compounds of formulae XII, XIII, XV and XVI are hydrogen; wherein the leaving group ‘Y’ in the compounds of formula XIV is Cl or Br; and wherein the group ‘R’ in the compounds of formulae XIV and XV is tert-butyl,
(b) the protecting groups P₁ and P₂ in the compounds of formulae VII, XI, XIII, XV and XVI are methyl, benzyl, t-butyldimethylsilyl and acetyl; wherein the leaving group ‘X’ in the compounds of formula XII is Br; and wherein the leaving group ‘Y’ in the compounds of formula XIV is Br;
(c) the groups P₁ and P₂ together with the atoms to which they are attached form an isopropylidene ring; or,
(d) the substituted cyclopentanamine derivative of formula VII obtained is [3aR-(3aα,4α,6α,6aα)]-2-[[6-amino-2,2-dimethyl tetrahydro-4H-cyclopenta-1,3-dioxol-4-yl]oxy]-ethanol of formula VIIa (formula VII, wherein P₁ and P₂ together with the atoms to which they are attached form an isopropylidene ring): 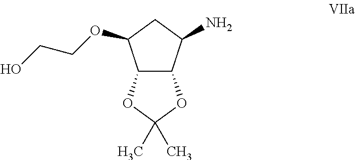
or an acid addition salt thereof

26-34. (canceled)

35. The process of claim 24, wherein the process is further defined according to one of (a), (b), or (c);
(a) the first solvent used in step-(a) is selected from the group consisting of water, a protic solvent, a solvent miscible with water, a dipolar aprotic solvent, and mixtures thereof; wherein the second solvent used in step-(b) is selected from the group consisting of acetone, methylethyl ketone, methylisobutyl ketone, methyltert-butyl ketone, acetonitrile, tetrahydrofuran, 2-methyl tetrahydrofuran, 1,4-dioxane, diethyl ether, diisopropyl ether, methyltert-butyl ether, monoglyme, diglyme, n-pentane, n-hexane, n-heptane, cyclohexane, toluene, xylene, N,N-dimethylformamide, N,N-dimethylacetamide, dimethylsulfoxide, N-methylpyrrolidone, and mixtures thereof; wherein the third solvent used in step-(c) is selected from the group consisting of a hydrocarbon, a cyclic ether, an aliphatic ether, a chlorinated hydrocarbon, and mixtures thereof; and wherein the fourth solvent used in step-(d) is selected from the group consisting of methanol, ethanol, isopropyl alcohol, n-propanol, n-butanol, tetrahydrofuran, 2-methyl tetrahydrofuran, 1,4-dioxane, diethyl ether, diisopropyl ether, methyl tert-butyl ether, dimethoxyethane, diethoxyethane, toluene, xylene, dichloromethane, dichloroethane, chloroform, and mixtures thereof;
(b) the first solvent is a mixture of water and ethanol; wherein the second solvent is N,N-dimethylformamide; wherein the third solvent is tetrahydrofuran; and wherein the fourth solvent used in step-(d) is selected from the group consisting of methanol, ethanol, 2-methyl tetrahydrofuran, tetrahydrofuran, and mixtures thereof;
(c) the alkylating agent used in step-(a) is benzyl bromide, benzyl chloride, a monosubstituted aralkyl halide or a polysubstituted aralkyl halide; and wherein the reducing agent used in step-(c) is selected from one of the groups (A) or (B):
(A) the group consisting of lithium aluminium hydride, lithium borohydride, sodium borohydride, borane, lithium tri-ter-butoxyaluminum hydride, borane-THF complex, diisobutylaluminum hydride (DIBAL-H), sodium bis(2-methoxyethoxy)aluminum hydride (Vitride®); or,
(B) the group consisting of diisobutylaluminum hydride (DIBAL-H) or sodium bis(2-methoxyethoxy)aluminum hydride (Vitride®) in toluene.

36-38. (canceled)

39. The process of claim 24, wherein the process is further defined according to (a) or (b):
(a) the reaction in step-(a) is optionally carried out via phase transfer catalysis wherein the amine to be protected and the nitrogen alkylating agent are reacted with a base in a solvent mixture in the presence of a phase transfer reagent, catalyst or promoter; and wherein the reaction in step-(b) is optionally carried out via phase transfer catalysis wherein the alcohol and the alkylating agent are reacted with a base in a solvent mixture in the presence of a phase transfer reagent, catalyst or promoter; or
(b) the deprotection in step-(d) is carried out by catalytic hydrogenation in the presence of a hydrogenation catalyst, optionally in the presence of an acid, under high pressure of about 40 to about 100 psi; or by catalytic transfer hydrogenation (CTH) in the presence of a catalytic transfer hydrogenation reagent, and optionally in the presence of an acid.

40. (canceled)

41. The process of claim 39, wherein the hydrogenation catalysts are Pd/C and Pd(OH)₂; wherein the acid is acetic acid; and wherein the catalytic transfer hydrogenation reagent is selected from the group consisting of 1,4-cyclohexadiene, cyclohexene, ammonium formate, formic acid, sodium formate, hydrazine, 1,3-cyclohexadiene and trialkylammonium formates, and combinations comprising the foregoing reagents.

42. A process for preparing a triazolo[4,5-d]pyrimidine compound of formula I: 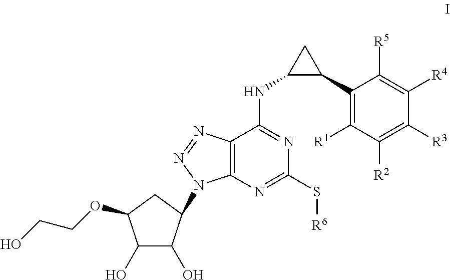
or a pharmaceutically acceptable salt thereof; wherein R¹, R², R³, R⁴ and R⁵ are, each independently, selected from hydrogen and a halogen atom, wherein the halogen atom is F, Cl, Br or I; and R⁶ is C₁₋₆ alkyl; comprising:
a) reacting the triazolo compound of formula X 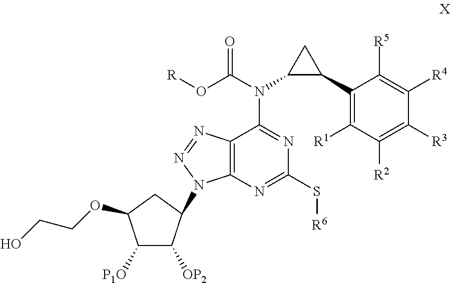
wherein R, R¹, R², R³, R⁴, R⁵, R⁶ and P¹ and P² are as defined above,
with a deprotecting agent in a first solvent to form a compound of formula XVII 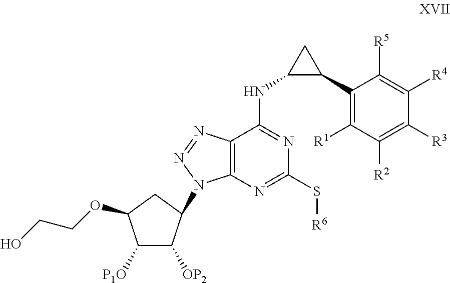
b) reacting a compound of formula XVII with an amino protecting group, in a second solvent and in presence of a base to produce a compound of formula XVIII 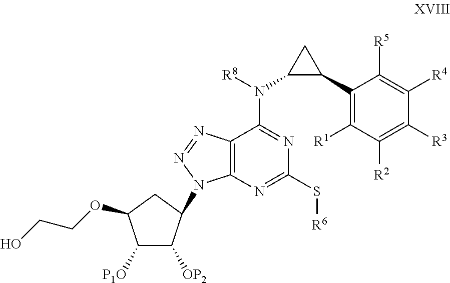
wherein R⁸ is a protecting group
c) reacting the compound of formula XVIII with an acid in a third solvent to produce a compound of formula XIX 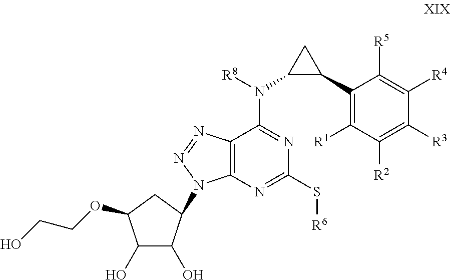
d) treating the compound of formula XIX with a deprotecting agent in a fourth solvent to produce a compound of formula I and optionally converting the compound of formula I into a pharmaceutically acceptable salt.

43. The process of claim 42, wherein the protecting groups P₁ and P₂ are defined according to one of (a), (b) or (c):
(a) the protecting groups P₁ and P₂ in the compounds of formulae X, XVII and XVIII are C₁₋₆ alkyl, benzyl, (C₁₋₆ alkyl)₃Si, and C(O)C₁₋₆ alkyl; wherein the groups R¹, R², and R⁵ in the compounds of formulae X, XVII, XVIII and XIX are hydrogen and R³ and R⁴ are halogen;
(b) the protecting groups P₁ and P₂ in the compounds of formulae X, XVII and XVIII are methyl, benzyl, t-butyldimethylsilyl and acetyl; or
(c) the groups P₁ and P₂ together with the atoms to which they are attached form an isopropylidene ring.

44.-49. (canceled)

50. The process of claim 42 wherein the process is further defined according to one of (a), (b), (c), (d), (e), (f), or (g):
(a) the deprotection agent used in step (a) is Iodine and the first solvent used in step (a) is selected from the group consisting of a ketone, an aliphatic or alicyclic hydrocarbon, a chlorinated aliphatic or aromatic hydrocarbon, an aromatic mono or dinitro hydrocarbon, an aliphatic or cyclic ether, a polar aprotic solvent, and mixtures thereof; wherein the second solvent used in step (b) is selected from hydrocarbon, ketones, ethers, aliphatic alcohol and mixtures thereof; more specifically the solvent used is acetone; wherein the third solvent used in step (c) is selected from an alcohol, ketone, a hydrocarbon, aliphatic ether, chlorinated hydrocarbon and mixtures thereof;
(b) the protecting agent used in step (b) is selected from C₁₋₆alkyl, benzyl, substituted benzyl (C₁₋₆ alkyl)₃Si and a C(O)C₁₋₆alkyl group and the base used in step (b) is selected from potassium carbonate, sodium carbonate, and lithium carbonate;
(c) the acid used in step (c) is selected from hydrochloric acid, hydrobromic acid, sulfuric acid, acetic acid, propionic acid, butanoic acid, pivalic acid, pentanoic acid, hexanoic acid, methane sulfonic acid, and mixtures thereof;
(d) the pH of the reaction mixture of step (c) is adjusted between 6-10 with an aqueous base;
(e) the pH of the reaction mixture of step (c) is adjusted to 10 with potassium carbonate;
(f) the deprotection in step (d) is carried out either by catalytic hydrogenation in the presence of a hydrogenation catalyst or by the catalytic transfer hydrogenation reagent; or
(g) the deprotection in step (d) is carried out using 10% palladium on carbon and formic acid in ethanol.

57. A process for preparing a triazolo[4,5-d]pyrimidine compound of formula I: 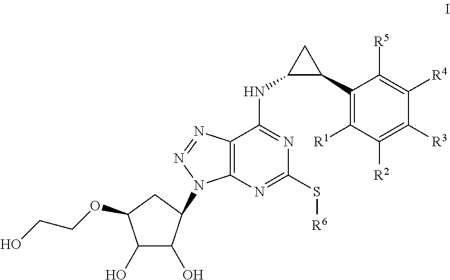
or a pharmaceutically acceptable salt thereof; wherein R¹, R², R³, R⁴ and R⁵ are, each independently, selected from hydrogen and a halogen atom, wherein the halogen atom is F, Cl, Br or I; and R⁶ is C₁₋₆ alkyl; comprising:
a) reacting a triazolo compound of formula X 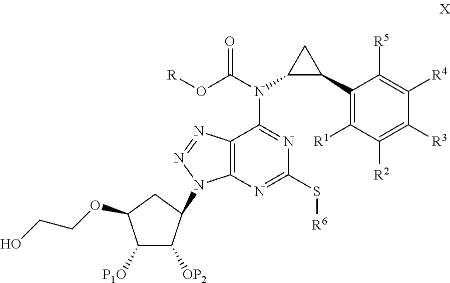
wherein R¹, R², R³, R⁴, R⁵, R⁶ and P₁ and P₂ are as defined above,
with an BOC anhydride in presence of a base to produce a compound of formula XX 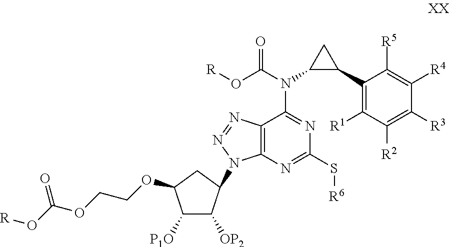
b) subjecting the compound of formula XX to acid hydrolysis or hydrogenolysis with an acid in a solvent to produce compound of formula I and optionally converting the compound of formula I into a pharmaceutically acceptable salt.

58-59. (canceled)

60. The process of claim 57, wherein the process is further defined according to one of (a), (b), (c), or (d):
(a) wherein the solvent used in step (b) is selected from a ketone, an aliphatic or alicyclic hydrocarbon, a chlorinated aliphatic or aromatic hydrocarbon, an aliphatic or cyclic ether, a polar aprotic solvent, and mixtures thereof;
(b) the acids used in step (b) is selected from hydrochloric acid, hydrobromic acid, sulfuric acid, acetic acid, propionic acid, butanoic acid, pivalic acid, pentanoic acid, hexanoic acid, methane sulfonic acid, and mixtures thereof;
(c) the pH of the reaction mixture of step (b) is adjusted between 6-10 with an aqueous base; or
(d) the pH of the reaction mixture of step (b) is adjusted to 10 with potassium carbonate.

61-63. (canceled)
